(19) United States
(12) Patent Application Publication
(10) Pub. No.: US 2009/0061422 A1
(43) Pub. Date: Mar. 5, 2009

(54) Diagnostic markers of breast cancer treatment and progression and methods of use thereof

(76) Inventors: Steven P. Linke, La Jolla, CA (US); Troy M. Bremer, La Jolla, CA (US); Cornelius A. Diamond, La Jolla, CA (US)

Correspondence Address: William C. Fuess, 10951 Sorrento Valley Road 2G, San Diego, CA, 92121, US

(21) Appl. No.: 11/787,518

(22) Filed: Apr. 17, 2007

Related U.S. Application Data
(63) Continuation-in-part of application No. 11/407,169, filed on Apr. 18, 2006.
(60) Provisional application No. 60/673,223, filed on Apr. 19, 2005.

(51) Int. Cl. C12Q 1/68; C12Q 1/02; G06G 7/60

(52) U.S. Cl. 435/6; 435/29; 703/11

(57) ABSTRACT

To maximize both the life expectancy and quality of life of patients with operable breast cancer, it is important to predict adjuvant treatment outcome and likelihood of progression before treatment. The instant invention details the usage of a machine-learning based method to develop a cross-validated model to predict the outcome of adjuvant treatment, particularly chemotherapy treatment outcome, and likelihood of progression before treatment. The model includes standard clinicopathological features, as well as molecular markers collected using standard immunohistochemistry and fluorescence in situ hybridization. The model significantly outperformed the St. Gallen Consensus guidelines and the Nottingham Prognostic Index and has the potential to provide a clinically useful and cost-effective prognostic for breast cancer patients.

### REFERENCE TO RELATED PATENT APPLICATIONS

The present application is a continuation-in-part of U.S. patent application Ser. No. 11/407,169, which application is descended from, and claims benefit of priority of, U.S. provisional patent application No. 60/673,223, filed Apr. 19, 2005. The related predecessor applications are hereby incorporated by reference in their entirety.

### GOVERNMENT SUPPORT

The present invention was developed under Research Support of the National Science Foundation, Award #0611297. The U.S. Government may have certain rights in this invention.

### FIELD OF THE INVENTION

The present invention generally pertains to the prediction of the outcome of adjuvant therapy in the treatment of breast cancer based on the presence and quantities of certain protein molecular markers, called biomarkers, present in the treated patients. The present invention also pertains to the prediction of progression of breast cancer, e.g. whether or not the patient's tumour is likely to metastasize, based upon cancer based on the presence and quantities of certain protein molecular markers.

The present invention specifically concerns the identification of groups, or “panels”, of biomarkers particularly useful in combination for enhanced predictive accuracy of patient response to breast cancer therapy with endocrine therapy, chemotherapy, targeted therapy, surgical-only resection with no drug therapy and/or radiation treatment, or a combination of these treatments.

### BACKGROUND OF THE INVENTION

The following discussion of the background of the invention is merely provided to aid the reader in understanding the invention and is not admitted to describe or constitute prior art to the present invention.

Breast cancer is the leading cause of cancer-related deaths in women worldwide (See for instance World Health Organization. Cancer. 2006). In the United States, estimates for 2007 indicate that over 240,000 new cases of in situ and invasive breast cancer will be diagnosed among women, and an additional 2,030 new cases will be diagnosed among men, and it is expected that over 40,000 women and 450 men will die from the disease this year (See for instance American Cancer Society. Cancer Facts and FIGS. 2007).

Roughly 75% of breast cancers are positive for the hormone-based estrogen receptor (ER) and/or progesterone receptor (PGR) (See for instance Osborne CK: Steroid hormone receptors in breast cancer management. Breast Cancer Res Treat 51:227-238, 1998). Most of these patients are treated with an endocrine therapy, either as an adjuvant to surgery in early stage disease or as the primary treatment in more advanced disease. The most common endocrine therapy has been the selective estrogen receptor modulator (SERM) tamoxifen (Nolvadex). It has been in use for over 20 years and demonstrably prolongs survival (See for instance Tamoxifen for early breast cancer: an overview of the randomised trials. Early Breast Cancer Trialists' Collaborative Group. Lancet 351:1451-1467, 1998).

Binding of estrogen to ER causes its phosphorylation and dimerization, followed by movement into the nucleus and transcription of a variety of genes, including secreted growth and angiogenic factors (See for instance Osborne C K, Shou J, Massarweh S, et al: Crosstalk between estrogen receptor and growth factor receptor pathways as a cause for endocrine therapy resistance in breast cancer. Clin Cancer Res 11:865s-870s, 2005 (suppl)), in a process called nuclear-initiated steroid signalling. There is also evidence of a membrane-bound fraction of ER that can activate other growth pathways, including the EGFR (ERBB1) and ERBB2 pathways (See for instance Shou J, Massarweh S, Osborne C K, et al: Mechanisms of tamoxifen resistance: increased estrogen receptor-HER2/neu cross-talk in ER/HER2-positive breast cancer. J Natl Cancer Inst 96:926-935, 2004), in a process called membrane-initiated steroid signalling. In breast tissue, tamoxifen competes with estrogen for binding to ER, thereby reducing proliferation through inhibition of ER's nuclear function. However, it also has been reported that tamoxifen can produce a weak agonist effect by stimulating the membrane-initiated signalling pathway when the relevant growth factors (e.g., EGFR and/or ERBB2) are overexpressed and/or by stimulating the nuclear-initiated pathway in the presence of overexpressed coactivators (e.g., NCOA1 and/or NCOA3) (See for instance Smith C L, Nawaz Z, O'Malley B W: Coactivator and corepressor regulation of the agonist/antagonist activity of the mixed antiestrogen, 4-hydroxytamoxifen. Mol Endocrinol 11:657-666, 1997; Osborne C K, Bardou V, Hopp T A, et al: Role of the estrogen receptor coactivator AIB1 (SRC-3) and HER-2/neu in tamoxifen resistance in breast cancer. J Natl Cancer Inst 95:353-361, 2003). Additional mechanisms of cross-talk between the growth factor receptor pathways may also lead to tamoxifen resistance (See for instance Clarke R, Liu M C, Bouker K B, et al: Antiestrogen resistance in breast cancer and the role of estrogen receptor signalling. Oncogene 22:7316-7339, 2003). In fact, approximately 40% of hormone receptor-positive patients fail to respond to tamoxifen (See for instance Nicholson R I, Gee J M, Knowiden J, et al: The biology of antihormone failure in breast cancer. Breast Cancer Res Treat 80 Suppl 1:S29-34; discussion S35, 2003 (suppl); Clarke R, Liu M C, Bouker K B, et al., Ibid.).

Related to these agonistic effects, tamoxifen can have a growth stimulatory effect on tissues such as the endometrium, leading to increased risk of endometrial hyperplasia and cancer. Other side effects include deep venous thrombosis and pulmonary emboli, development of benign ovarian cysts, vaginal discharge or irritation and hot flashes, and vision problems. There is also evidence of increased risk of gastrointestinal cancer and stroke (See for instance Breast cancer (PDQ): Ibid.).

In the past 25 years, several generations of adjuvant chemotherapy regimens have been developed. These changes have led to gradual improvements in treatment efficacy, but have also resulted in more-prolonged therapy, added toxicity and substantially increased costs. Meta-analysis of randomized adjuvant chemotherapy trial data has shown that the regimen of cyclophosphamide, methotrexate and 5-fluorouracil (CMF) decreased the annual breast cancer death rate by around 34% in women aged less than 50 years and by 10% in women aged 50-69 years (for instance see Early Breast Cancer Trialists' Collaborative Group (EBCTCG) (2005) Effects of chemotherapy and hormonal therapy for early breast cancer on recurrence and 15-year survival: an overview of the randomised trials. Lancet 365: 1687-1717). The absolute improvements in survival are usually much smaller and depend on the absolute risk of recurrence. Seventeen trials that included 14,470 patients have also directly compared CMF with anthracycline-based adjuvant chemotherapy. Meta-analysis of these results has shown that inclusion of an anthracycline further decreased the annual breast cancer death rate by around 16% compared with CMF alone. The long-term toxicities from anthracyclines, however, include secondary leukemia (cumulative incidence at 5 years<0.1-0.6%) and left ventricular dysfunction (1-2%) (for instance see Doyle J J et al. (2005) Chemotherapy and cardiotoxicity in older breast cancer patients: a population-based study. J Clin Oncol 23: 8597-8605). More recently, addition of taxanes to anthracycline-based regimens was evaluated and showed a small but consistent further improvement in disease-free survival (for instance see Trudeau M et al. (2005) Selection of adjuvant chemotherapy for treatment of node-positive breast cancer. Lancet Oncol 6: 886-898).

Intense research has been conducted in recent years on molecular markers that could provide prognostic information and/or predict treatment outcome. It will be seen that the study supportive of the present invention served to analyze data on the standard hormone receptors (ER and PGR), as well as the growth factor receptors EGFR and ERBB2. In addition, the tumour suppressors CDKN1B and TP-53, the anti-apoptotic factor BCL2, the proliferation markers CCND1 and KI-67, the cell-cycle marker CDKN1B, the MYC oncogene, and over 50 other markers were among those studied.

Although a number of studies have been published indicating that these markers have or likely have prognostic significance, some studies have not confirmed the findings, and no consensus has been reached on their utility. More importantly; however, the present invention will be seen to demonstrate the importance of the conditional interpretation of certain markers on others due to their interdependency. Some of these studies will be detailed in later sections of the instant invention.

Research specific to panels of molecular markers include U.S. patent application Ser. No. 11/037,713 which uses a subset of the dataset that is detailed in the Examples to claim several panels of markers involved in “ . . . detecting, diagnosing, staging, monitoring, predicting, preventing conditions associated with breast cancer.” However, said marker panels claimed do not use an algorithm to interpolate the sum total of effects, resulting in an AUC ROC of 0.74 versus 0.90 in the instant invention, said panels do not incorporate clinicopathological features such as stage and grade, and said panels require a minimum of 21 molecular markers to produce the preferred result, whereas the instant invention requires only seven. U.S. patent application Ser. No. 10/376,538, filed Mar. 1, 2003, specifies a ratio of the isoforms of the signaling protein Shc, e.g. PY-Shc to p66-Shc, differentiates between aggressive and non-aggressive breast cancer tumors. Ratios like these, as also demonstrated in U.S. patent application Ser. No. 10/727,100, generally do not separate patients any better than using just clinicopathological variable such as stage, grade, and ER status, and do not work in lymph-node negative patients (see for instance Jansen M et al., HOXB13-to-IL17BR expression ratio is related with tumor aggressiveness and response to tamoxifen of recurrent breast cancer, J Clin Oncol. 2007 Feb. 20; 25(6):662-8.). U.S. patent application Ser. No. 11/061,067 details several multi-marker panels that define patient outcome based upon “ . . . assessing the patient's likely prognosis based upon binding of the panel to the tumor sample.” This method is equivalent with a ‘voting scheme’ in which just the presence or absence of the binding of an antibody is enough to give a prognostic indication. However, as the instant invention describes below, the scheme detailed in U.S. patent application Ser. No. 11/061,067 is not enough to produce a diagnostic of sufficient sensitivity and specificity, as shown by the fact that the method described does not beat current prognostic indicators such as the Nottingham Prognostic Index or Adjuvant! Online (see for instance Eden P, Ritz C, Rose C, Ferno M, Peterson C: “Good Old” clinical markers have similar power in breast cancer prognosis as microarray gene expression profilers. Eur J Cancer 40:1837-41, 2004). Still other relevant literature to the instant invention include U.S. patent application Ser. Nos. 10/872,063, 10/883,303, and 10/852,797, which claim gene-expression tests for predicting breast cancer progression and treatment to various chemotherapies. As described below, gene expression tests have numerous problems in that the relevant genes that are claimed come from examining a number of samples x which is orders of magnitude less than the total number of genes y initially examined. In doing such, it is unlikely from a statistical viewpoint that such gene sets will produce the same sensitivity and specificity as the initial result detailed in U.S. patent application Ser. Nos. 10/872,063, 10/883,303, and 10/852,797, and other literature described elsewhere in the initial invention. U.S. patent application Ser. Nos. 10/872,063, 10/883,303, and 10/852,797 make mention of the protein products of such genes in producing such a test, but (1) this is not enabled in these patents and (2) the instant invention enables in its claims a minimal set of specific protein product biomarkers interpolated by a specific nonlinear algorithms which allows a highly sensitive and specific test validated by independent testing patient populations. Beyond the statistical issues, gene expression assays can only measure transcript levels, which do not always correlate with functional protein levels, and they cannot detect protein mislocalization. In addition, the assays are relatively complicated and costly, often requiring sophisticated and/or proprietary technology and multiple steps, including methods to try to reduce the contribution of adjacent non-tumor tissue and to account for RNA degradation.

In contrast, the present invention will be seen to concern the development of a multi-molecular marker diagnostic with significant contributions by ER, PGR, BCL2, ERBB2, CDKN1B, c-MYC, TP-53, and others, in addition to standard clinicopathological factors, all interpolated by an algorithm that can deliver superior prognostic ability as compared to individual protein markers or gene expression techniques.

### BRIEF SUMMARY OF THE INVENTION

The present invention is embodied in a method of providing a prognosis of disease-free survival in a cancer patient comprising the steps of obtaining a sample from the patient; and then determining various polypeptide levels (e.g. molecular markers) in the sample, wherein change in various polypeptide levels as compared to a control sample indicates the good prognosis of a prolonged disease-free survival. The present invention contemplates a multiple molecular marker diagnostic, the values of each assayed marker collectively interpolated by a non-linear algorithm, to (1) predict the outcomes of endocrine, particularly tamoxifen, therapy for breast cancer in consideration of multiple molecular makers, called biomarkers, of a patient's; and (2) identify whether or not a tumour from a patient is likely to be more aggressive, or malignant, than another and thus requiring neoadjuvant chemotherapy in addition to surgical and radiological treatment. The model was built by multivariate mathematical analysis of (1) many more multiple molecular markers, called biomarkers, than ultimately proved to be significant in combination for forecasting treatment outcomes, in consideration of (2) real-world, clinical, outcomes of real patients who possessed these biomarkers.

The diagnostic is subject to updating, or revision, as any of (1) new biomarkers are considered, (2) new patient data (including as may come from patients who had their own treatment outcomes predicted) becomes available, and/or (3) new (drug) therapies are administered, all without destroying the validity of the instant invention and of discoveries made during the building, and the exercise, thereof, as hereinafter discussed.

A number of different insights are derived from the (1) building and (2) the exercise of the diagnostic. A primary insight may be considered to be the identification of a number, or a “palette”, of biomarkers that are in combination of superior, and even greatly superior, accuracy for use in predicting the outcomes of tamoxifen therapy for breast cancer than would be any one, or even two, markers taken alone or in a ratio. This predictive power of the combination over that of a simple voting panel is increased by use of an algorithm that interpolates the linear and non-linear collective contributions of said combination in order to predict the clinical outcome of interest.

A secondary insight from the diagnostic is that certain biomarkers are of increased accuracy in predicting, in particular, percentage disease-specific survival at 30+ months from onset of treatment of breast cancer when these biomarkers taken in pairs. This does not mean that these biomarker pairs are of overall predictive accuracy equal or superior to the palette of predictive biomarkers. It only means that, when considered in pairs, certain biomarkers provide useful subordinate predictions.

Finally, a tertiary insight that falls out from the identification of biomarker pairs having superior predictive accuracy is that expected disease-specific survival can, and does, vary greatly when, sometimes, but one single one of these biomarkers changes, as during the course of the treatment of single patient.

### THEORY OF THE INVENTION

In accordance with the present invention, exercise of the diagnostic primarily serves to (1) identify and quantify a palette of biomarkers interpolated by a non-linear algorithm having superior predictive capability for prognosis of outcomes in endocrine therapy of breast cancer; and (2) determine which patients would benefit from adjuvant chemotherapy and/or targeted therapy.

In another of its aspects, the instant invention is embodied in methods for choosing one or more marker(s) for diagnosis, prognosis, or therapeutic treatment of breast cancer in a patient that together, and as a group, have maximal sensitivity, specificity, and predictive power. Said maximal sensitivity, specificity, and predictive power is in particular realized by choosing one or more markers as constitute a group by a process of plotting receiver operator characteristic (ROC) curves for (1) the sensitivity of a particular combination of markers versus (2) specificity for said combination at various cutoff threshold levels. In addition, the instant invention further discloses methods to interpolate the nonlinear correlative effects of one or more markers chosen by any methodology to such that the interaction between markers of said combination of one or more markers promotes maximal sensitivity, specificity, and predictive accuracy in the diagnosis, prognosis, or therapeutic treatment of breast cancer.

In various aspects, the present invention relates to (1) materials and procedures for identifying markers that are associated with the diagnosis, prognosis, or differentiation of breast cancer in a patient; (2) using such markers in diagnosing and treating a patient and/or monitoring the course of a treatment regimen; (3) using such markers to identify subjects at risk for one or more adverse outcomes related to breast cancer; and (4) using at one of such markers an outcome marker for screening compounds and pharmaceutical compositions that might provide a benefit in treating or preventing such conditions.

The first three aspects of the present invention are discussed in the following sections below.

A Palette of Biomarkers Relevant to the Prognosis of Outcome in therapeutic treatment of Breast Cancer

A diagnostic assay relating diverse biomarkers to real-world, clinical, outcomes from endocrine therapy of breast cancer having being built, optimised and exercised by the present invention as hereinafter explained, a specific palette of molecular markers, also called biomarkers, useful in predicting outcomes to endocrine therapy in the treatment of breast cancer patients has been identified.

The preferred predictive palette was derived from a multivariate mathematical model where over fifty-six (56) biomarkers were taken into consideration, and where seven (7) such biomarkers were found to be of improved prognostic significance taken in combination. Specifically, the most preferred palette of biomarkers predictive of outcome in adjuvant therapy for breast cancer include the protein expression of ER, PGR, BCL2, ERBB2, TP-53, CDKN1B, and c-MYC gene amplification.

Pair-wise, as well as multivariate, dependence of certain biomarkers

Second, in accordance with the present invention an interdependency of certain biomarkers, and groups of biomarkers, has been recognised. Historically at least one dependency has been suggested, such as by anecdotal evidence it has been known that the unitary predictive value of the ER biomarker is influenced by the expression level of the PGR biomarker. However, the present invention reveals new interdependencies, and even usefully quantifies these dependencies to show the varying predictive value of one biomarker in consideration of another.

Use of an algorithm in combining the effects of several markers to predict response to therapy.

Provided in the present invention is a method of providing a treatment decision for a cancer patient receiving endocrine, chemo- and/or targeted therapy comprising obtaining a sample from the patient; and determining various molecular marker levels of interest in the sample, inputting such values into an algorithm which has previously correlated in a machine-learning fashion relationships between said molecular marker levels and clinical outcome, wherein output from such an algorithm indicates that that the cancer is a type of cancer that would be treatable with the selected treatment regime.

Thus, in certain embodiments of the methods of the present invention, a plurality of markers and clinicopathological factors are combined using an algorithm to increase the predictive value of the analysis in comparison to that obtained from the markers taken individually or in smaller groups. Most preferably, one or more markers for adhesion, angiogenesis, apoptosis, catenin, catenin/cadherin proliferation/differentiation, cell cycle, cell-cell interactions, cell-cell movement, cell-cell recognition, cell-cell signalling, cell surface, centrosomal, cytoskeletal, ERBB2 interaction, growth factors, growth factor receptors, invasion, metastasis, membrane/integrin, oncogenes, proliferation, tumour suppression, signal transduction, surface antigen, transcription factors and specific and non-specific markers of breast cancer are combined in a single assay to enhance the predictive value of the described methods. This assay is usefully predictive of multiple outcomes, for instance: diagnosis of breast cancer, then predicting breast cancer prognosis, then further predicting response to treatment outcome. Moreover, different marker combinations in the assay may be used for different indications. Correspondingly, different algorithms interpret the marker levels as indicated on the same assay for different indications.

In preferred embodiments, particular thresholds for one or more molecular markers in a panel are not relied upon to determine if a profile of marker levels obtained from a subject are indicative of a particular diagnosis/prognosis. Rather, in accordance with the present invention, an evaluation of the entire profile is made by (1) first training an algorithm with marker information from samples from a test population and a disease population to which the clinical outcome of interest has occurred to determine weighting factors for each marker, and (2) then evaluating that result on a previously unseen population. Certain persons skilled in bioinformatics will recognise this procedure to be tantamount to the construction, and to the training, of a neural network. The evaluation is determined by maximising the numerical area under the ROC curve for the sensitivity of a particular panel of markers versus specificity for said panel at various individual marker levels. From this number, the skilled artisan can then predict a probability that a subject's current marker levels in said combination is indicative of the clinical marker of interest. For example, (1) the test population might consist solely of samples from a group of subjects who have survived over 10 years after treatment of their breast cancer with adjuvant chemotherapy and no other comorbid disease conditions, while (2) the disease population might consist solely of samples from a group of subjects who have had breast cancer and treated such cancer with endocrine therapy only, and have no other comorbid disease conditions. A third, “normal” population might also be used to establish baseline levels of markers as well in a non-diseased population.

In preferred embodiments of the marker, and marker panel, selection methods of the present invention, the aforementioned weighting factors are multiplicative of marker levels in a non-linear fashion. Each weighting factor is a function of other marker levels in the panel combination, and consists of terms that relate individual contributions, or independent and correlative, or dependent, terms. In the case of a marker having no interaction with other markers in regards to then clinical outcome of interest, then the specific value of the dependent terms would be zero.

### Other Embodiments of the Instant Invention

In another embodiment of the instant invention, the response to therapy is a complete pathological response.

In a preferred embodiment, the subject is a human patient.

If the tumor is breast cancer, it can, for example, be invasive breast cancer, or stage II or stage III breast cancer.

In a specific embodiment of the invention, the patient is not receiving an endocrine therapy, a chemotherapy, a targeted therapy or another hormonal therapy. In another embodiment, the patient is concurrently receiving an endocrine therapy, chemotherapy or a hormonal therapy. In a specific embodiment, the endocrine therapy comprises tamoxifen, raloxifene, megestrol, or toremifene. In a further specific embodiment, the targeted therapy comprises lapitinab, bevacizumab, trastuzumab, cetuximab, or panitumumab. In a further specific embodiment, another hormonal therapy is an aromatase inhibitor such as anastrozole, letrozole, or exemestane, or pure anti-estrogens such fulvestrant, or surgical or medical means (goserelin, leuprolide) for reducing ovarian function. In a further specific embodiment, the cancer comprises an estrogen receptor-positive cancer or a progesterone receptor-positive cancer.

In a particular embodiment, the chemotherapy is adjuvant or neoadjuvant chemotherapy.

The neoadjuvant chemotherapy may, for example, comprise the administration of a taxane derivative, such as docetaxel and/or paclitaxel, and/or other anti-cancer agents, such as, members of the anthracycline class of anti-cancer agents, doxorubicin, topoisomerase inhibitors, etc.

The method may involve determination of the expression levels of at least two, or at least three, or at least four, or at least 5, or at least 6, or at least 7, or at least 8, or at least 9, or at least 10, or at least 15, or at least 20 of the prognostic proteins listed within this specification, listed above, or their associative protein expression products, as well as one or more gene amplifications of the markers listed within this specification.

The biological sample may be e.g. a tissue sample comprising cancer cells, where the tissue can be fixed, paraffin-embedded, or fresh, or frozen.

In a particular embodiment, the tissue is from fine needle, core, or other types of biopsy.

In another embodiment, the tissue sample is obtained by fine needle aspiration, bronchial lavage, or transbronchial biopsy.

The expression level of said prognostic protein levels or associated protein levels can be determined, for example, by immunohistochemistry or a western blot, or other proteomics techniques, or any other methods known in the art, or their combination.

In an embodiment, the assay for the measurement of said prognostic proteins or their associated expression products is provided is provided in the form of a kit or kits for staining of individual proteins upon sections of tumor tissue.

In another embodiment, said kit is designed to work on an automated platform for analysis of cells and tissues such as described in U.S. patent application Ser. No. 10/062,308 entitled ‘Systems and methods for automated analysis of cells and tissues’.

An embodiment of the invention is a method of screening for a compound that improves the effectiveness of a said adjuvant therapy in a patient comprising the steps of: introducing to a cell a test agent, wherein the cell comprises polynucleotide(s) mentioned in the instant invention encoding polypeptide(s) under control of a promoter operable in the cell; and measuring said polypeptide level(s), wherein when the level(s) are decreased following the introduction, the test agent is the compound that improves effectiveness of said adjuvant therapy in the patient. It is also contemplated that such an agent will prevent the development of said adjuvant therapy resistance in a patient receiving such a therapy. In a specific embodiment, the patient is said adjuvant therapy-resistant. It is also contemplated that the test agent is a ribozyme, an antisense nucleotide, a receptor blocking antibody, a small molecule inhibitor, or a promoter inhibitor.

An embodiment of the invention is a method of screening for a compound that improves the effectiveness of a said adjuvant therapy in a patient comprising the steps of: contacting a test agent with polypeptide(s) mentioned in the instant invention, wherein said polypeptide(s) or the ER polypeptide is linked to a marker; and determining the ability of the test agent to interfere with the binding of said polypeptide(s), wherein when the marker level(s) are decreased following the contacting, the test agent is the compound that improves effectiveness of the adjuvant therapy in the patient. In certain embodiments of the invention, the patient is adjuvant therapy-resistant for said adjuvant therapy.

One embodiment of the invention is a method of treating a cancer patient comprising administering to the patient a therapeutically effective amount of an antagonist of polypeptide(s) mentioned in the instant invention and an said adjuvant therapy. In certain embodiments of the invention, the patient is said adjuvant therapy-resistant. A specific embodiment of the invention is presented wherein the antagonist interferes with translation of the polypeptide(s) mentioned in the instant invention. In a further specific embodiment of the invention the antagonist interferes with an interaction between the polypeptide(s) mentioned in the instant invention and an estrogen receptor polypeptide. The antagonist interferes with phosphorylation or any other posttranslational modification of the said polypeptide(s) in yet another specific embodiment of the invention. In another specific embodiment of the invention, the antagonist inhibits the function of a polypeptide encoding a kinase that specifically phosphorylates said polypeptide(s). In another embodiment, the antagonist is administered before, together with, or after the said adjuvant therapy. The antagonist and the said adjuvant therapy are administered at the same time in another embodiment.

An embodiment of the invention is method of improving the effectiveness of a said adjuvant therapy in a cancer patient comprising administering a therapeutically effective amount of an antagonist of polypeptide level (s) mentioned in the instant invention to the patient to provide a therapeutic benefit to the patient. In a specific embodiment, the administering is systemic, regional, local or direct with respect to the cancer.

An embodiment of the invention is a method of determining whether a pre-menopausal breast cancer patient should have ovariectomy as a treatment option (also goserulin, leupitine, letrozole, exesmestane, anastrozole, fulvestrant). Elevated levels of polypeptide(s) mentioned in the instant invention in a tumor sample are indicative of ovariectomy as a possible treatment option.

An embodiment of the invention is a method of determining whether a cancer patient has de novo endocrine therapy resistance comprising the steps of: obtaining a sample from the patient; and determining polypeptide(s) mentioned in the instant invention in the sample and a HER-2 polypeptide level in the sample, wherein elevated polypeptide(s) mentioned in the instant invention as compared to a control sample indicate de novo endocrine therapy resistance.

Other embodiments, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is Table 1 of patient characteristics of the Tristar and IPC dataset and Table 2 of numbers and outcomes of the patients studied in the IPC dataset.

FIG. 2 is Table 3 of molecular markers studied.

FIG. 3 is Table 4 of IHC marker utility analysis for several additional markers in assay models described in various examples and Table 5 of Multivariate Cox Proportional hazard analysis of a model described in the examples and its relevance in certain patient subsets whose prognosis is determined by current clinical guidelines.

FIG. 4 is comprised of graphs showing the previously published model in the IPC dataset. FIG. 4a shows a Kaplan-Meier survival curve for overall survival. FIG. 4b shows the effect of chemotherapy on the good prognosis group as defined by the previously published model.

FIG. 5 is comprised of graphs showing Kaplan-Meier survival curves in the IPC dataset. FIG. 5a shows the effect of chemotherapy on the good prognosis group as defined by the previously published model. FIG. 5b shows the effect of chemotherapy on all HR positive patients who received hormone therapy.

FIG. 6 is comprised of gra/phs showing Kaplan-Meier survival curves of model 1D in the IPC dataset. FIG. 6a is model 1D applied to HR positive untreated patients. FIG. 6b is model 1D applied to HR-positive patients treated with chemotherapy only.

FIG. 7 is comprised of graphs showing Kaplan-Meier survival curves of model 1D in the IPC dataset. FIG. 7a is model 1D applied to HR-positive patients treated with endocrine therapy only. FIG. 7b is model 1D applied to HR-positive patients treated with chemotherapy and endocrine therapy.

FIG. 8 is comprised of graphs showing Kaplan-Meier survival curves of model 1D in the IPC dataset. FIG. 8a shows the chemotherapy benefit in the poor prognosis group of HR-positive patients. FIG. 8b shows model 1D separating patients not treated with hormone therapy in the NPI-bad (NPI>3.4) category.

FIG. 9 is comprised of graphs showing Kaplan-Meier survival curves of model 1D in the IPC dataset. FIG. 9a shows model 1D separating patients treated with hormone therapy in the NPI-bad (NPI>3.4) category. FIG. 9b shows model 1D the chemotherapy benefit in patients treated with hormone therapy in the NPI-bad (NPI>3.4) category.

FIG. 10 is comprised of graphs showing Kaplan-Meier survival curves of model 1D in the IPC dataset. FIG. 10a shows model 1D separating patients not treated with hormone therapy in the St. Gallen's intermediate and high-risk categories. FIG. 10b shows model 1D separating patients treated with hormone therapy in the St. Gallen's intermediate and high-risk categories.

FIG. 11 is comprised of graphs showing Kaplan-Meier survival curves of model 1D in the IPC dataset. FIG. 11a shows the chemotherapy benefit of model 1D in the St. Gallen's intermediate and high-risk categories. FIG. 11b shows continuous decrease in risk of death based upon a risk score given by model 1D.

FIG. 12 is a look-up table to which the interpolative algorithm behind model 1D can be reduced to.

FIG. 13 is a look-up table of the adjusted risk using Adjuvant! Online. As noted in these tables, an adjustment for pN>0 still remains in the biomarker risk score as in the original model. The model may be operated either with or without the adjustment. To use the model without the adjustment simply use the pN=0 Tables.

### DETAILED DESCRIPTION OF THE INVENTION

### Definitions

As used herein, the term “adjuvant” refers to a pharmacological agent that is provided to a patient as an additional therapy to the primary treatment of a disease or condition.

The term “algorithm” as used herein refers to a mathematical formula that provides a relationship between two or more quantities. Such a formula may be linear, non-linear, and may exist as various numerical weighting factors in computer memory.

The term “control sample” as used herein indicates a sample that is compared to a patient sample. A control sample may be obtained from the same tissue that the patient sample is taken from. However, a noncancerous area may be chosen to reflect the individual polypeptide levels in normal cells for a particular patient. A control may be a cell line, such as MCF-7, in which serial dilutions are undertaken to determine the exact concentration of elevated polypeptide levels. Such levels are compared with a patient sample. A “control sample” may comprise a theoretical patient with an elevated polypeptide level of a certain molecule that is calculated to be the cutoff point for elevated polypeptide levels of said certain molecule. A patient sample that has polypeptide levels equal to or greater than such a control sample is said to have elevated polypeptide levels.

As used herein, the term “overall survival” is defined to be survival after first diagnosis and death. For instance, long-term overall survival is for at least 5 years, more preferably for at least 8 years, most preferably for at least 10 years following surgery or other treatment.

The term “disease-free survival” as used herein is defined as a time between the first diagnosis and/or first surgery to treat a cancer patient and a first reoccurrence. For example, a disease-free survival is “low” if the cancer patient has a first reoccurrence within five years after tumor resection, and more specifically, if the cancer patient has less than about 55% disease-free survival over 5 years. For example, a high disease-free survival refers to at least about 55% disease-free survival over 5 years.

The term “endocrine therapy” as used herein is defined as a treatment of or pertaining to any of the ducts or endocrine glands characterized by secreting internally and into the bloodstream from the cells of the gland. The treatment may remove the gland, block hormone synthesis, or prevent the hormone from binding to its receptor.

The term “adjuvant therapy-resistant patient” as used herein is defined as a patient receiving an endocrine therapy and lacks demonstration of a desired physiological effect, such as a therapeutic benefit, from the administration of an adjuvant therapy.

The term “estrogen-receptor positive” as used herein refers to cancers that do have estrogen receptors while those breast cancers that do not possess estrogen receptors are “estrogen receptor-negative.”

The term “polypeptide” as used herein is used interchangeably with the term “protein”, and is defined as a molecule which comprises more than one amino acid subunits. The polypeptide may be an entire protein or it may be a fragment of a protein, such as a peptide or an oligopeptide. The polypeptide may also comprise alterations to the amino acid subunits, such as methylation or acetylation. The term “molecular marker” is also used interchangeably with the terms protein and polypeptide, though the two latter terms are subclasses of the former.

The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “prognosis” as used herein are defined as a prediction of a probable course and/or outcome of a disease. For example, in the present invention the combination of several protein levels together with an interpolative algorithm constitutes a prognostic model for determination of survival outcome in a cancer patient.

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

The term “therapeutic benefit” as used herein refers to anything that promotes or enhances the well-being of the subject with respect to the medical treatment of his condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition. In a specific embodiment, a therapeutic benefit refers to reversing de novo adjuvant therapy-resistance or preventing the patient from acquiring an adjuvant therapy-resistance.

The term “therapeutically effective amount” as used herein is defined as the amount of a molecule or a compound required to improve a symptom associated with a disease. For example, in the treatment of cancer such as breast cancer, a molecule or a compound which decreases, prevents, delays or arrests any symptom of the breast cancer is therapeutically effective. A therapeutically effective amount of a molecule or a compound is not required to cure a disease but will provide a treatment for a disease. A molecule or a compound is to be administered in a therapeutically effective amount if the amount administered is physiologically significant. A molecule or a compound is physiologically significant if its presence results in technical change in the physiology of a recipient organism.

The term “treatment” as used herein is defined as the management of a patient through medical or surgical means. The treatment improves or alleviates at least one symptom of a medical condition or disease and is not required to provide a cure. The term “treatment outcome” as used herein is the physical effect upon the patient of the treatment.

The term “sample” as used herein indicates a patient sample containing at least one tumor cell. Tissue or cell samples can be removed from almost any part of the body. The most appropriate method for obtaining a sample depends on the type of cancer that is suspected or diagnosed. Biopsy methods include needle, endoscopic, and excisional. The treatment of the tumor sample after removal from the body depends on the type of detection method that will be employed for determining individual protein levels.

### DETAILED DESCRIPTION

We previously developed a preliminary model to predict outcome in hormone receptor (HR)-positive, hormone therapy (HT)-treated stage I-III breast cancer patients (Linke et al., U.S. patent application Ser. No. 11/407,169) (henceforth “preliminary published model”). The model was trained and tested using robust cross-validation on a dataset with demographic, treatment, and outcome data, as well as data on a number of standard clinicopathologic features and nine molecular markers (TriStar dataset). The TriStar dataset included 324 patients treated adjuvantly with HT (>98% tamoxifen) with or without locoregional radiotherapy (RT), but not cytotoxic chemotherapy (CT). It also included an additional 103 patients who received both HT and CT on which no models have been trained.

The preliminary published model included six of the molecular markers plus age≧85 years and pathological lymph node status (pN). The molecular markers were ER, PGR, ERBB2, BCL2, and TP53 assessed by immunohistochemistry (IHC), and MYC assessed by fluorescence in situ hybridization (FISH). Thresholds for each of the selected features were chosen from the available continuous or categorical data. This preliminary published model, as well as all of the models described herein, generate a risk score for each patient that is directly associated with risk of death. A risk score cut-off of −0.31 was used to classify individual patients into good and poor prognosis categories, which accurately predicted their outcome (Linke et al., A multi-marker model to predict outcome in tamoxifen-treated breast cancer patients. Clin Cancer Res 12:1175-1183, 2006).

Accordingly, the instant invention provides an expanded panel model that incorporates the marker CDKN1B (hereafter CDKN1B) and additional markers, is determined by an algorithm whose weights for individual marker interactions relating to outcome can be reduced to a look-up table, and is shown to be beneficial in predicting likely survival for various time-periods when chemotherapy is given alone or in concert with an endocrine therapy.

Methodology of Marker Selection, Analysis, and Classification

A comprehensive methodology for identification of one or more markers for the prognosis, diagnosis, and detection of breast cancer has been described previously in U.S. patent application Ser. No. 11/407,169, entitled DIAGNOSTIC MARKERS OF BREAST CANCER TREATMENT AND PROGRESSION AND METHODS OF USE THEREOF, filed Apr. 18, 2006, the patents referred to within and parent applications are hereby incorporated by reference in its entirety, including all tables, figures, and claims. Our method of predicting relevant markers given an individual's test sample is an automated technique of constructing an optimal mapping between a given set of input marker data and a given clinical variable of interest and is now explained in brief.

We first obtain patient test samples of tissue from two or more groups of patients. The patients are those exhibiting symptoms of a disease event, say breast cancer, and who are prescribed a specific therapeutic treatment which has a specific clinical outcome are compared to a different set of patients also exhibiting the same disease event but with different therapeutic treatments and/or clinical outcome of said treatment. These second sets of patients are viewed as controls, though these patients might have another disease event distinct from the first. Samples from these patients are taken at various time periods after the event has occurred, and assayed for various markers as described within. Clinicopathological information, such as age, tumor stage, tumor histological grade, and node status are collected at time of diagnosis. These markers and clinicopathological information form a set of examples of clinical inputs and their corresponding outputs, the outputs being the clinical outcome of interest, for instance breast cancer prognosis and/or breast cancer therapeutic treatment outcome.

We then use an algorithm to select the most relevant clinical inputs that correspond to the outcome for each time period. This process is also known as feature selection. In this process, the minimum number of relevant clinical inputs, e.g. features, that are needed to fully differentiate and/or predict disease prognosis, diagnosis, or detection with the highest sensitivity and specificity are selected for each time period. The feature selection is done with an algorithm that selects markers that differentiate between patient disease groups, say those likely to have recurrence versus those likely to no recurrence. The relevant clinical input combinations might change at different time periods, and might be different for different clinical outcomes of interest.

We then train a classifier to map the selected relevant clinical inputs to the outputs. A classifier assigns relative weightings to individual marker values. We note that the construct of a classifier is not crucial to our method. Any mapping procedure between inputs and outputs that produces a measure of goodness of fit, for example, maximizing the area under the receiver operator curve of sensitivity versus 1-specificity, for the training data and maximizes it with a standard optimization routine on a series of validation sets would also suffice.

Once the classifier is trained, it is ready for use by a clinician. The clinician enters the same classifier inputs used during training of the network by assaying the selected markers and collecting relevant clinical information for a new patient, and the trained classifier outputs a maximum likelihood estimator for the value of the output given the inputs for the current patient. The clinician or patient can then act on this value. We note that a straightforward extension of our technique could produce an optimum range of output values given the patient's inputs as well as specific threshold values for inputs.

One versed in the ordinary state of the art knows that many other polypeptides in the literature once measured from tumor tissue in a diseased patient and healthy tissue from a healthy patient, selected through use of an feature selection algorithm might be prognostic of breast cancer or breast cancer treatment outcome if measured in combination with others and evaluated together with a nonlinear classification algorithm. A list of some of these other polypeptides, previously considered for diagnosis or prognosis of breast cancer and thus not novel in themselves, has been previously detailed in U.S. patent application Ser. No. 11/407,169. This list is meant to serve as illustrative and not meant to be exhaustive. Selected polypeptide descriptions in said list may be similar to U.S. patent application Ser. No. 10/758,307, U.S. patent application Ser. No. 11/061,067 and/or U.S. patent application Ser. No. 10/872,063, all of which are noted as prior art. However, the instant invention goes beyond what is taught or anticipated in these applications, providing a rigorous methodology of discovering which representative polypeptides are best suited to building a predictive model for determining a clinical outcome and building a model for interpolating between such polypeptides in conjunction with clinicopathological variables to determine clinical outcome, while the methodology described in U.S. patent application Ser. No. 10/758,307, U.S. patent application Ser. No. 11/061,067 and/or U.S. patent application Ser. No. 10/872,063 rely on simple linear relationships between markers and linear optimization techniques to find them. Using such techniques, the instant invention also defines smaller, more robust sets of polypeptides that are more predictive of clinical outcome than what is described or anticipated in such applications.

Method for Defining Panels of Markers

In practice, data may be obtained from a group of subjects. The subjects may be patients who have been tested for the presence or level of certain polypeptides and/or clinicopathological variables (hereafter ‘markers’ or ‘biomarkers’). Such markers and methods of patient extraction are well known to those skilled in the art. A particular set of markers may be relevant to a particular condition or disease. The method is not dependent on the actual markers. The markers discussed in this document are included only for illustration and are not intended to limit the scope of the invention. Examples of such markers and panels of markers are described in U.S. patent application Ser. No. 11/407,169 and the incorporated references.

Well-known to one of ordinary skill in the art is the collection of patient samples. A preferred embodiment of the instant invention is that the samples come from two or more different sets of patients, one a disease group of interest and the other(s) a control group, which may be healthy or diseased in a different indication than the disease group of interest. For instance, one might want to look at the difference in markers between patients who have had endocrine therapy and had a recurrence of cancer within a certain time period and those who had endocrine therapy and did not have recurrence of cancer within the same time period to differentiate between the two populations.

When obtaining tumor samples for testing according to the present invention, it is generally preferred that the samples represent or reflect characteristics of a population of patients or samples. It may also be useful to handle and process the samples under conditions and according to techniques common to clinical laboratories. Although the present invention is not intended to be limited to the strategies used for processing tumor samples, we note that, in the field of pathology, it is often common to fix samples in buffered formalin, and then to dehydrate them by immersion in increasing concentrations of ethanol followed by xylene. Samples are then embedded into paraffin, which is then molded into a “paraffin block” that is a standard intermediate in histologic processing of tissue samples. The present inventors have found that many useful antibodies to biomarkers discussed herein display comparable binding regardless of the method of preparation of tumor samples; those of ordinary skill in the art can readily adjust observations to account for differences in preparation procedure.

In preferred embodiments of the invention, large numbers of tissue samples are analyzed simultaneously. In some embodiments, a tissue array is prepared. Tissue arrays may be constructed according to a variety of techniques. According to one procedure, a commercially-available mechanical device (e.g., the manual tissue arrayer MTAL from Beecher Instruments of Sun Prairie, Wis.) is used to remove an 0.6-micron-diameter, full thickness “core” from a paraffin block (the donor block) prepared from each patient, and to insert the core into a separate paraffin block (the recipient block) in a designated location on a grid. In preferred embodiments, cores from as many as about 400 patients can be inserted into a single recipient block; preferably, core-to-core spacing is approximately 1 mm. The resulting tissue array may be processed into thin sections for staining with interaction partners according to standard methods applicable to paraffin embedded material. Depending upon the thickness of the donor blocks, as well as the dimensions of the clinical material, a single tissue array can yield about 50-150 slides containing>75% relevant tumor material for assessment with interaction partners. Construction of two or more parallel tissue arrays of cores from the same cohort of patient samples can provide relevant tumor material from the same set of patients in duplicate or more. Of course, in some cases, additional samples will be present in one array and not another.

The tumor test samples are assayed by one or more techniques, well-known for those versed in ordinary skill in the art for various polypeptide levels. Briefly, assays are conducted by binding a certain substance with a detectable label to the antibody of the protein in question to be assayed and bringing such in contact with the tumor sample to be assayed. Any available technique may be used to detect binding between an interaction partner and a tumour sample. One powerful and commonly used technique is to have a detectable label associated (directly or indirectly) with the antibody. For example, commonly-used labels that often are associated with antibodies used in binding studies include fluorochromes, enzymes, gold, iodine, etc. Tissue staining by bound interaction partners is then assessed, preferably by a trained pathologist or cytotechnologist. For example, a scoring system may be utilised to designate whether the antibody to the polypeptide does or does not bind to (e.g., stain) the sample, whether it stains the sample strongly or weakly and/or whether useful information could not be obtained (e.g., because the sample was lost, there was no tumor in the sample or the result was otherwise ambiguous). Those of ordinary skill in the art will recognise that the precise characteristics of the scoring system are not critical to the invention. For example, staining may be assessed qualitatively or quantitatively; more or less subtle gradations of staining may be defined; etc.

It is to be understood that the present invention is not limited to using antibodies or antibody fragments as interaction partners of inventive tumour markers. In particular, the present invention also encompasses the use of synthetic interaction partners that mimic the functions of antibodies. Several approaches to designing and/or identifying antibody mimics have been proposed and demonstrated (e.g., see the reviews by Hsieh-Wilson et al., Acc. Chem. Res. 29:164, 2000 and Peczuh and Hamilton, Chem. Rev. 100:2479, 2000). For example, small molecules that bind protein surfaces in a fashion similar to that of natural proteins have been identified by screening synthetic libraries of small molecules or natural product isolates (e.g., see Gallop et al., J. Med. Chem. 37:1233, 1994; Gordon et al., J. Med. Chem. 37:1385, 1994; DeWitt et al., Proc. Natl. Acad. Sci. U.S.A. 90:6909, 1993; Bunin et al., Proc. Natl. Acad. Sci. U.S.A. 91:4708, 1994; Virgilio and Ellman, J. Am. Chem. Soc. 116:11580, 1994; Wang et al., J. Med. Chem. 38:2995, 1995; and Kick and ElIman, J. Med. Chem. 38:1427, 1995). Similarly, combinatorial approaches have been successfully applied to screen libraries of peptides and polypeptides for their ability to bind a range of proteins (e.g., see Cull et al., Proc. Natl. Acad. Sci. U.S.A. 89:1865, 1992; Mattheakis et al., Proc. Natl. Acad. Sci. U.S.A. 91:9022, 1994; Scott and Smith, Science 249:386, 1990; Devlin et al., Science 249:404, 1990; Corey et al., Gene 128:129, 1993; Bray et al., Tetrahedron Lett. 31:5811, 1990; Fodor et al., Science 251:767, 1991; Houghten et al., Nature 354:84, 1991; Lam et al., Nature 354:82, 1991; Blake and Litzi-Davis, Bioconjugate Chem. 3:510, 1992; Needels et al., Proc. Natl. Acad. Sci. U.S.A. 90:10700, 1993; and Ohimeyer et al., Proc. Natl. Acad. Sci. U.S.A. 90:10922, 1993). Similar approaches have also been used to study carbohydrate-protein interactions (e.g., see Oldenburg et al., Proc. Natl. Acad. Sci. U.S.A. 89:5393, 1992) and polynucleotide-protein interactions (e.g., see Ellington and Szostak, Nature 346:818, 1990 and Tuerk and Gold, Science 249:505, 1990). These approaches have also been extended to study interactions between proteins and unnatural biopolymers such as oligocarbamates, oligoureas, oligosulfones, etc. (e.g., see Zuckermann et al., J. Am. Chem. Soc. 114:10646, 1992; Simon et al., Proc. Natl. Acad. Sci. U.S.A. 89:9367, 1992; Zuckermann et al., J. Med. Chem. 37:2678, 1994; Burgess et al., Angew. Chem., Int. Ed. EngI. 34:907, 1995; and Cho et al., Science 261:1303, 1993). Yet further, alternative protein scaffolds that are loosely based around the basic fold of antibody molecules have been suggested and may be used in the preparation of inventive interaction partners (e.g., see Ku and Schultz Proc. Natl. Acad. Sci. U.S.A. 92:6552, 1995). Antibody mimics comprising a scaffold of a small molecule such as 3-aminomethylbenzoic acid and a substituent consisting of a single peptide loop have also been constructed. The peptide loop performs the binding function in these mimics (e.g., see Smythe et al., J. Am. Chem. Soc. 116:2725, 1994). A synthetic antibody mimic comprising multiple peptide loops built around a calixarene unit has also been described (e.g., see U.S. Pat. No. 5,770,380 to Hamilton et al.).

Any available strategy or system may be utilised to detect association between an antibody and its associated polypeptide molecular marker. In certain embodiments, association can be detected by adding a detectable label to the antibody. In other embodiments, association can be detected by using a labeled secondary antibody that associates specifically with the antibody, e.g., as is well known in the art of antigen/antibody detection. The detectable label may be directly detectable or indirectly detectable, e.g., through combined action with one or more additional members of a signal producing system. Examples of directly detectable labels include radioactive, paramagnetic, fluorescent, light scattering, absorptive and calorimetric labels. Examples of indirectly detectable include chemiluminescent labels, e.g., enzymes that are capable of converting a substrate to a chromogenic product such as alkaline phosphatase, horseradish peroxidase and the like.

Once a labeled antibody has bound a tumor marker, the complex may be visualized or detected in a variety of ways, with the particular manner of detection being chosen based on the particular detectable label, where representative detection means include, e.g., scintillation counting, autoradiography, measurement of paramagnetism, fluorescence measurement, light absorption measurement, measurement of light scattering and the like.

In general, association between an antibody and its polypeptide molecular marker may be assayed by contacting the antibody with a tumor sample that includes the marker. Depending upon the nature of the sample, appropriate methods include, but are not limited to, immunohistochemistry (IHC), radioimmunoassay, ELISA, immunoblotting and fluorescence activates cell sorting (FACS). In the case where the polypeptide is to be detected in a tissue sample, e.g., a biopsy sample, IHC is a particularly appropriate detection method. Techniques for obtaining tissue and cell samples and performing IHC and FACS are well known in the art.

In general, the results of such an assay can be presented in any of a variety of formats. The results can be presented in a qualitative fashion. For example, the test report may indicate only whether or not a particular protein biomarker was detected, perhaps also with an indication of the limits of detection. Additionally the test report may indicate the subcellular location of binding, e.g., nuclear versus cytoplasmic and/or the relative levels of binding in these different subcellular locations. The results may be presented in a semi-quantitative fashion. For example, various ranges may be defined and the ranges may be assigned a score (e.g., 0 to 5) that provides a certain degree of quantitative information. Such a score may reflect various factors, e.g., the number of cells in which the tumor marker is detected, the intensity of the signal (which may indicate the level of expression of the tumor marker), etc. The results may be presented in a quantitative fashion, e.g., as a percentage of cells in which the tumor marker is detected, as a concentration, etc. As will be appreciated by one of ordinary skill in the art, the type of output provided by a test will vary depending upon the technical limitations of the test and the biological significance associated with detection of the protein biomarker. For example, in the case of certain protein biomarkers a purely qualitative output (e.g., whether or not the protein is detected at a certain detection level) provides significant information. In other cases a more quantitative output (e.g., a ratio of the level of expression of the protein in two samples) is necessary.

The resulting set of values are put into a database, along with outcome, also called phenotype, information detailing the treatment type, for instance tamoxifen plus chemotherapy, once this is known. Additional patient or tumour test sample details such as patient nodal status, histological grade, cancer stage, the sum total called patient clinicopathological information, are put into the database. The database can be simple as a spreadsheet, i.e. a two-dimensional table of values, with rows being patients and columns being filled with patient marker and other characteristic values.

From this database, a computerized algorithm can first perform pre-processing of the data values. This involves normalisation of the values across the dataset and/or transformation into a different representation for further processing. The dataset is then analysed for missing values. Missing values are either replaced using an imputation algorithm, in a preferred embodiment using KNN or MVC algorithms, or the patient attached to the missing value is excised from the database. If greater than 50% of the other patients have the same missing value then value can be ignored.

Once all missing values have been accounted for, the dataset is split up into three parts: a training set comprising 33-80% of the patients and their associated values, a testing set comprising 10-50% of the patients and their associated values, and a validation set comprising 1-50% of the patients and their associated values. These datasets can be further sub-divided or combined according to algorithmic accuracy. A feature selection algorithm is applied to the training dataset. This feature selection algorithm selects the most relevant marker values and/or patient characteristics. Preferred feature selection algorithms include, but are not limited to, Forward or Backward Floating, SVMs, Markov Blankets, Tree Based Methods with node discarding, Genetic Algorithms, Regression-based methods, kernel-based methods, and filter-based methods.

Feature selection is done in a cross-validated fashion, preferably in a nafve or k-fold fashion, as to not induce bias in the results and is tested with the testing dataset. Cross-validation is one of several approaches to estimating how well the features selected from some training data is going to perform on future as-yet-unseen data and is well-known to the skilled artisan. Cross validation is a model evaluation method that is better than residuals. The problem with residual evaluations is that they do not give an indication of how well the learner will do when it is asked to make new predictions for data it has not already seen. One way to overcome this problem is to not use the entire data set when training a learner. Some of the data is removed before training begins. Then when training is done, the data that was removed can be used to test the performance of the learned model on “new” data.

Once the algorithm has returned a list of selected markers, one can optimize these selected markers by applying a classifier to the training dataset to predict clinical outcome. A cost function that the classifier optimizes is specified according to outcome desired, for instance an area under receiver-operator curve maximising the product of sensitivity and specificity of the selected markers, or positive or negative predictive accuracy. Testing of the classifier is done on the testing dataset in a cross-validated fashion, preferably naïve or k-fold cross-validation. Further detail is given in U.S. patent application Ser. No. 09/611,220, incorporated by reference. Classifiers map input variables, in this case patient marker values, to outcomes of interest, for instance, prediction of stroke sub-type. Preferred classifiers include, but are not limited to, neural networks, Decision Trees, genetic algorithms, SVMs, Regression Trees, Cascade Correlation, Group Method Data Handling (GMDH), Multivariate Adaptive Regression Splines (MARS), Multilinear Interpolation, Radial Basis Functions, Robust Regression, Cascade Correlation+Projection Pursuit, linear regression, Non-linear regression, Polynomial Regression, Regression Trees, Multilinear Interpolation, MARS, Bayes classifiers and networks, and Markov Models, and Kernel Methods.

The classification model is then optimised by for instance combining the model with other models in an ensemble fashion. Preferred methods for classifier optimization include, but are not limited to, boosting, bagging, entropy-based, and voting networks. This classifier is now known as the final predictive model. The predictive model is tested on the validation data set, not used in either feature selection or classification, to obtain an estimate of performance in a similar population.

The predictive model can be translated into a decision tree format for subdividing the patient population and making the decision output of the model easy to understand for the clinician. The marker input values might include a time since symptom onset value and/or a threshold value. Using these marker inputs, the predictive model delivers diagnostic or prognostic output value along with associated error. The predictive model might be further reduced to a look-up table that can be stored in a database that details the outcome for markers in all possible states. The instant invention anticipates a kit comprised of reagents, devices and instructions for performing the assays, and a computer software program comprised of the predictive model that interprets the assay values when entered into the predictive model run on a computer. The predictive model receives the marker values via the computer that it resides upon.

Once patients are exhibiting symptoms of cancer, for instance breast cancer, a tissue tumor sample is taken from the patient using standard techniques well known to those of ordinary skill in the art and assayed for various tumor markers of cancer by slicing it along its radial axis and placing such slices upon a substrate for molecular analysis by assaying for various molecular markers. Assays can be preformed through immunohistochemistry or through any of the other techniques well known to the skilled artisan. In a preferred embodiment, the assay is in a format that permits multiple markers to be tested from one sample, such as the Aqua platform.™, and/or in a quantitative fashion, defined to within 10% of the actual value and in the most preferred enablement of the instant invention, within 1% of the actual value. The values of the markers in the samples are inputted into the trained, tested, and validated algorithm residing on a computer, which outputs to the user on a display and/or in printed format on paper and/or transmits the information to another display source the result of the algorithm calculations in numerical form, a probability estimate of the clinical diagnosis of the patient. There is an error given to the probability estimate, in a preferred embodiment this error level is a confidence level. The medical worker can then use this diagnosis to help guide treatment of the patient.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses. Marker antibodies or antigens may be incorporated into immunoassay diagnostic kits depending upon which marker autoantibodies or antigens are being measured. A first container may include a composition comprising an antigen or antibody preparation. Both antibody and antigen preparations should preferably be provided in a suitable titrated form, with antigen concentrations and/or antibody titers given for easy reference in quantitative applications.

The kits may also include an immunodetection reagent or label for the detection of specific immunoreaction between the provided antigen and/or antibody, as the case may be, and the diagnostic sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction is detected or quantified by means of detecting or quantifying the label. Immunodetection reagents and processes suitable for application in connection with the novel methods of the present invention are generally well known in the art.

The reagents may also include ancillary agents such as buffering agents and protein stabilizing agents, e.g., polysaccharides and the like. The diagnostic kit may further include where necessary agents for reducing background interference in a test, agents for increasing signal, software and algorithms for combining and interpolating marker values to produce a prediction of clinical outcome of interest, apparatus for conducting a test, calibration curves and charts, standardization curves and charts, look-up tables, and the like.

Various aspects of the invention may be better understood in view of the following detailed descriptions, examples, discussion, and supporting references.

### EXAMPLES

### Example I

### Derivation of, and Conclusions from, the Invention in Brief

We previously developed a preliminary model to predict outcome in hormone receptor (HR)-positive, hormone therapy (HT)-treated stage I-III breast cancer patients (see Linke et al: A multi-marker model to predict outcome in tamoxifen-treated breast cancer patients. Clin Cancer Res 12:1175-1183, 2006, henceforth “preliminary published model”). The model was trained and tested using robust cross-validation on a dataset, hereafter called the TriStar dataset, with demographic, treatment, and outcome data, as well as data on a number of standard clinicopathologic features and nine molecular markers. The TriStar dataset included 324 patients treated adjuvantly with HT (>98% tamoxifen) with or without locoregional radiotherapy (RT), but not cytotoxic chemotherapy (CT). It also included an additional 103 patients who received both HT and CT on which no models have been trained.

The preliminary published model included six of the molecular markers plus age≧85 years and pathological lymph node status (pN). The molecular markers were ER, PGR, ERBB2, BCL2, and TP53 assessed by immunohistochemistry (IHC), and MYC assessed by fluorescence in situ hybridization (FISH). Thresholds for each of the selected features were chosen from the available continuous or categorical data. This preliminary published model, as well as all of the models described herein, generate a risk score for each patient that is directly associated with risk of death. A risk score cut-off of −0.31 was used to classify individual patients into good and poor prognosis categories, which accurately predicted their outcome.

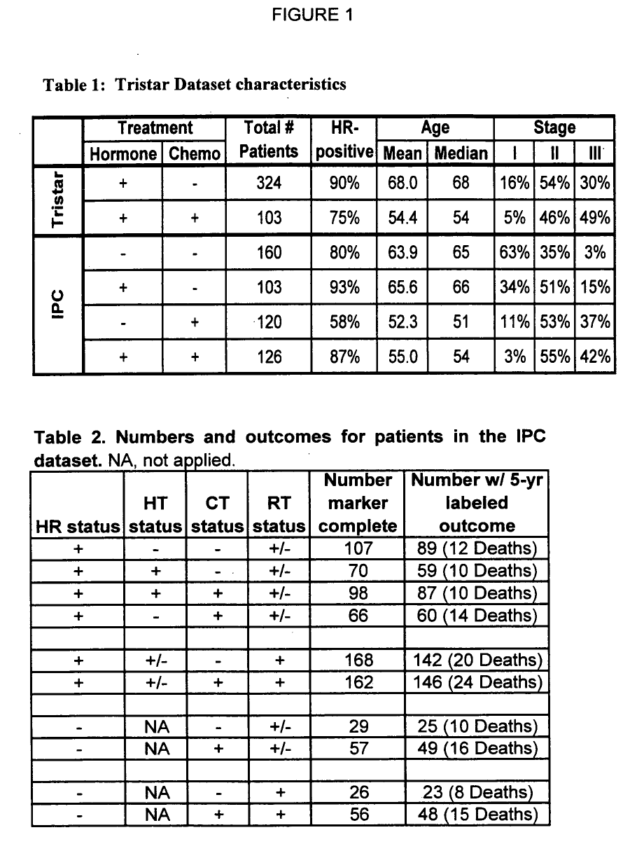

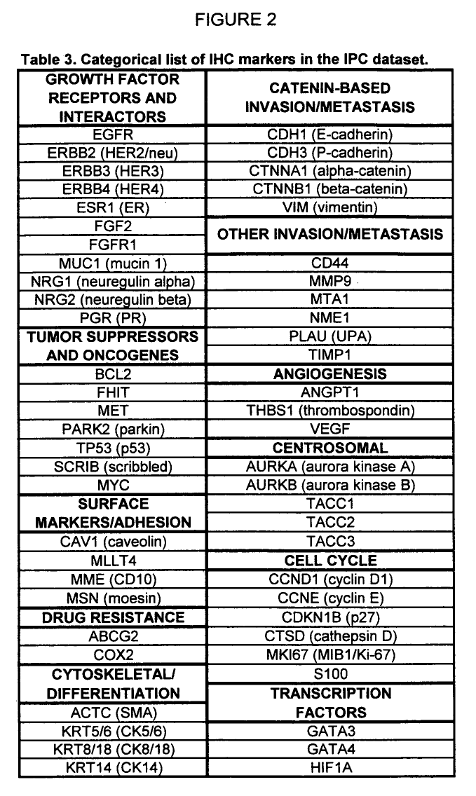

An independent dataset was obtained from the Institut Paoli-Calmette, Marseille, France, (hereafter the IPC dataset) in which we obtained demographic, treatment, outcome, clinicopathologic, and molecular data on an independent set of 547 patients. The IPC dataset included a total of 56 molecular markers selected based on their reported predictive or prognostic role in breast cancer, including the five IHC markers in our preliminary published model. Molecular marker scoring was done semi-quantitatively by pathologists, similar to the TriStar dataset, with proportion (range=0-100%) and intensity (range=0-3) scores multiplied to produce composite scores (range=0-300). Characterization of the IPC dataset, including methods for assaying the proteomic markers, has been detailed in U.S. patent application Ser. No. 11/037,713, filed Jan. 18, 2005. Table 1 (FIG. 1) gives some comparison statistics relevant to the instant invention between the two datasets. Table 2 (FIG. 2) gives numbers and outcomes relevant to the instant invention.

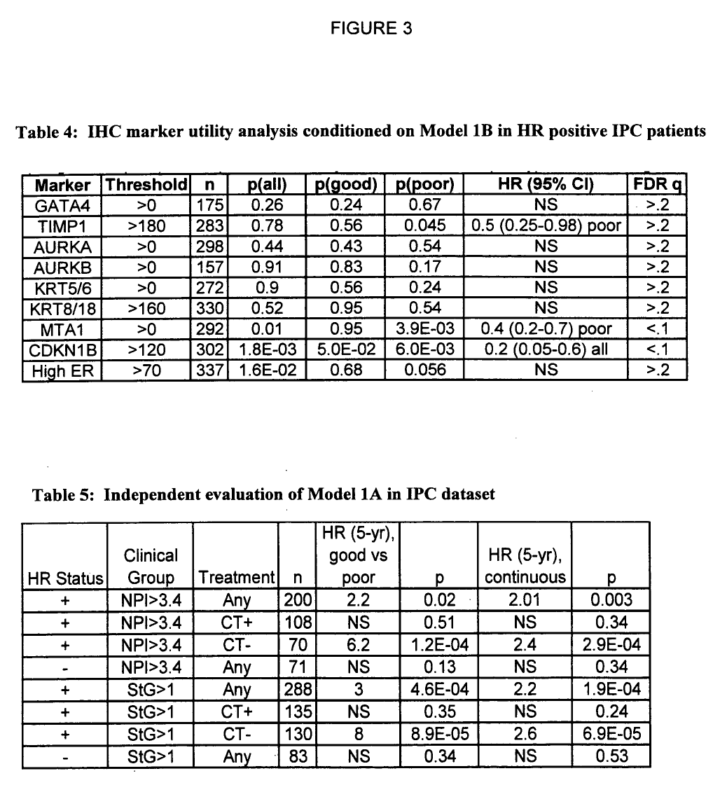

Table 3 (FIG. 3) provides a categorical list of the 56 IHC markers in the IPC dataset. The categories are only general, because many of the markers appear to play multiple roles, and some have currently ill-defined roles. Many of the markers center on the ER signaling pathway and the complex cross-talk that occurs with alternative growth factor receptor pathways. A number of the markers are involved in proliferation, cell division, differentiation, apoptosis, adhesion, invasion, or angiogenesis pathways, and several are transcription factors, tumor suppressors, or oncogenes. For comprehensive reviews of breast cancer markers for prognosis and prediction, see for instance Coradini D, Daidone MG: Biomolecular prognostic factors in breast cancer. Curr Opin Obstet Gynecol 16:49-55, 2004; Esteva F J, Hortobagyi G N: Prognostic molecular markers in early breast cancer. Breast Cancer Res 6:109-18, 2004; and Ross J S, et al: Breast cancer biomarkers and molecular medicine: part II. Expert Rev Mol Diagn 4:169-88, 2004. With regard to HT or CT treatment resistance, the markers may play direct roles, indirect roles (general prognostic indicators), or both.

Extensive research in breast cancer has shown that, while single markers can be statistically significant, it is unlikely that a single or only a few markers can effectively predict patient outcome with acceptable confidence. Markers involved in the biological pathways central to tumorigenesis and drug response exhibit higher-order nonlinear dependencies and interactions. Thus, the task of identifying the features (demographic, clinicopathologic, and/or molecular) that perform well in an algorithm that classifies patient outcome is a difficult one, and the choice of a high-performing set of features can be non-intuitive. For example, molecular markers that perform poorly separately can often achieve better performance when paired with other molecular markers or clinicopathologic characteristics. To uncover collective predictive value hidden to linear correlations with single markers and conventional biostatistics, we used a “statistical pattern recognition” approach. This included machine learning technology to identify and weight features to accurately predict patient outcome.

### Example II

Independent validation of the 6-biomarker tamoxifen treatment outcome model.

First, the full IPC dataset was pre-processed/coded into a fully numeric state for analysis.

Second, the data distributions (labeled based on patient outcome) of the five IHC markers in the preliminary published model were compared to those in the HT-only subset of the IPC dataset. This was done to verify that the methods used to score these markers (i.e., proportion of positive cells, staining intensity, etc.) yielded comparable distributions to those observed in the TriStar dataset. All five of the IHC markers were deemed acceptable for model application, as their scored distributions were similar, and their hazard ratios, when stratified by key clinicopathogic factors, were not statistically different between the two datasets. Data for these markers also was sufficiently complete in the patients to allow survival analysis. The threshold values used for the five IHC markers in the IPC dataset were equivalent to those used to produce the preliminary published model in the TriStar dataset. The remaining molecular marker, MYC amplification by FISH, was determined to be a poor prognostic indicator that was largely independent of all of the other features in the original TriStar dataset, and it affected a relatively small subset of patients (˜11%). Based on these observations, and because reliable MYC FISH data was not available in the IPC dataset, all of the IPC patients were assumed to be negative for MYC amplification.

Third, robust boot-strapped parameter estimation was conducted on the features identified in the preliminary published model within the original TriStar dataset to develop a model (henceforth “Model 1A”) for independent validation in the IPC dataset. The features included the five IHC markers (ER, PGR, ERBB2, BCL2, and TP53), as well as pN and age≧85 years (see Linke et al Ibid.). Model 1A was developed using a third-order polynomial kernel partial least squares (KPLS) method. Risk scores were calculated for the patients in the IPC dataset using Model 1A, and they were classified as good or poor prognosis. Identical to the preliminary published model, a risk score threshold of −0.31 was used to classify patients into good and poor prognosis categories (good prognosis, ≦−0.31; poor prognosis, >−0.31).

Survival analysis, in combination with Cox proportional hazards analysis, was used to assess the performance and significance of the model. FIG. 1 shows that Model 1A provided a strong and highly statistically significant discrimination of the good and poor prognosis groups in the HR-positive, non-CT-treated IPC patients. The good prognosis group had a 5-year overall survival (OS) approximating the expected survival of a similar non-cancer population, as calculated in R using the “survexp” routine based on an age- and sex-matched general mortality rate from 1960-1980 United States census records. These results are statistically equivalent to those achieved with our preliminary published model (see Linke et al Ibid.), providing a fully independent validation.

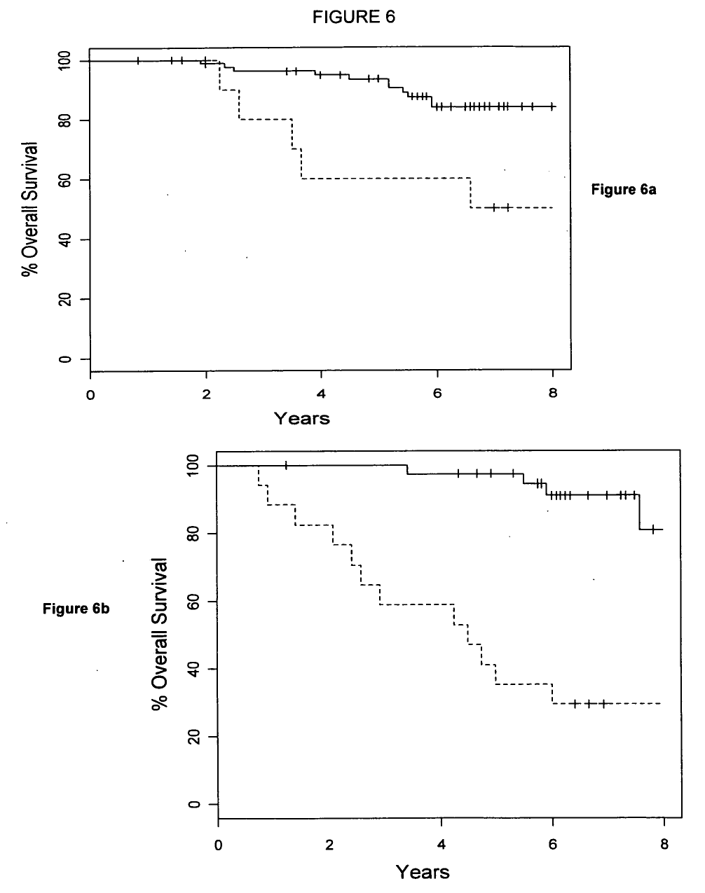

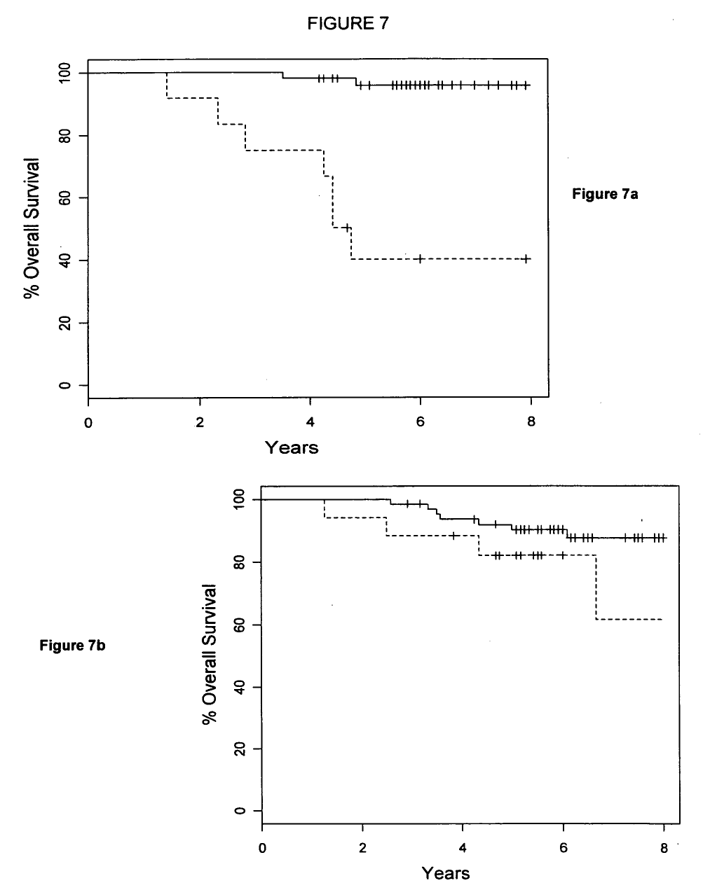

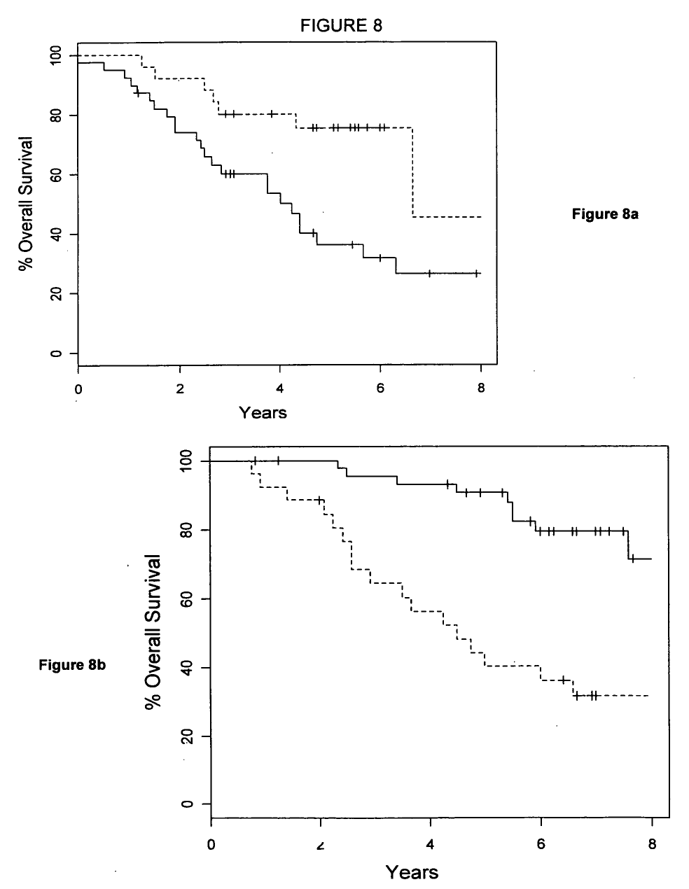

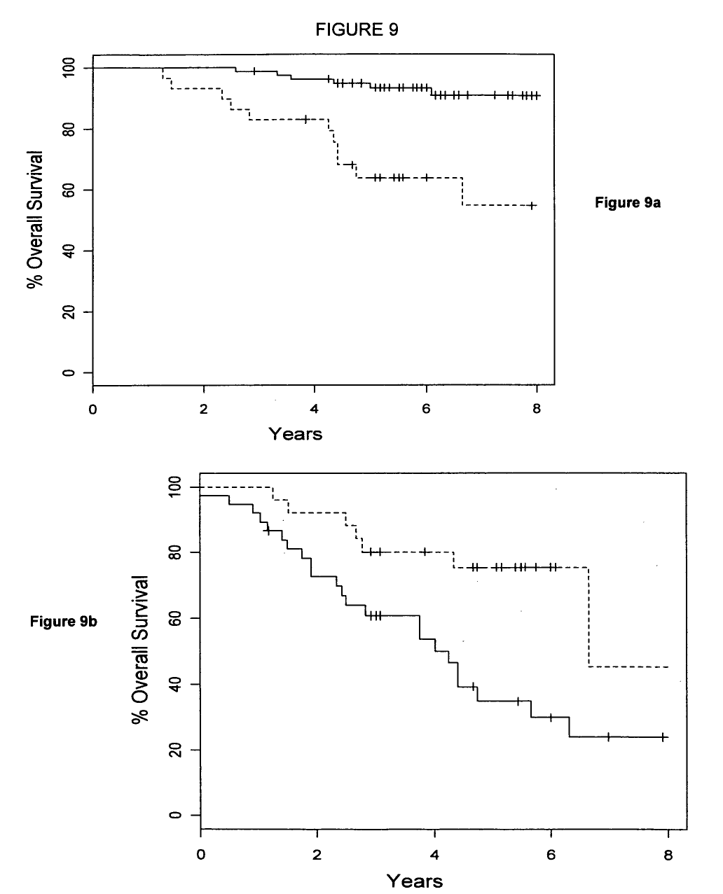

We then extended the application of Model 1A to the HR-positive, CT-treated IPC patients. FIG. 7 shows that the good prognosis group treated with CT had a survival that was equivalent to the non-CT-treated good prognosis group shown in FIG. 6. This indicates that patients in our good prognosis group do not derive a benefit from CT, suggestive of over-treatment. In contrast, FIG. 7 shows that the poor prognosis group treated with CT had a substantial benefit relative to the non-CT-treated poor prognosis group shown in FIG. 6. FIG. 8 directly shows the benefit of CT in the poor prognosis population. Interestingly, the survival benefit imparted by CT in the overall HR-positive, HT-treated IPC population did not reach statistical significance (FIG. 9). In combination, these data indicate that Model 1A is effective at identifying patients who are at risk of death without more aggressive adjuvant therapy (poor prognosis), and that the poor prognosis patients have an elevated chance of benefit from CT.

Fourth, an executable program was developed to allow easy application of the outcome model to new datasets. The parameters for the features in the preliminary published model were transferred to the executable after being robustly estimated using repeated application of five-fold cross-validation on the original TriStar dataset. The parameter estimates were stored in a data file compliant with the executable program to enable consistent application of the model (henceforth “Model 1B”). The executable program reads in the raw continuous or categorical data for each of the model features and applies the same thresholds that were used in the original published study. The program also codes any missing data to allow consistent application of the model. As described above for data distribution testing, patients missing MYC FISH data are assumed to be negative for MYC amplification by the program.

### Example III

Biological informatics-based development of a multimarker model to predict outcome in patients treated with hormone therapy and/or chemotherapy.

We next sought to improve our prognostic models by data mining the entire IPC set, including all 56 IHC markers and the associated clinicopathologic data in patients within all treatment groups. A wrapper-based feature selection approach, sequential forward floating feature selection (SFFS), was used within a nested, stratified cross-validation schema in conjunction with multiple machine learning algorithms. These methods identified the markers most relevant to prognosis (dimensionality reduction) and jointly optimized the parameters. They comprise a comprehensive non-linear model approach to achieve high classification accuracy.

Nested, stratified cross-validation: During feature selection and modeling, the data was broken up into different sets: training/validation and testing. The training/validation sets are the complement to the disjoint testing sets, which are naïve to model optimization and feature selection. A model is optimized for each putative feature combination on the training set, and then the feature set/model is scored using the validation component of the training set to allow robust feature selection. The models form nonlinear “maps” of the effects of various factors upon the clinical outcome of interest from the input data. The cross-validation during feature selection avoids overfitting (i.e., memorization of features in a specific dataset that are not applicable in a general manner).

SFFS: SFFS is an excellent wrapper-based, sub-optimal search procedure with lower computational cost than the gold standard “branch and bound” procedure. It is similar to sequential forward search, but it provides for removal of features in an evolving feature set when it improves classifier performance (see Pudil P, Novovicov J, Kittier J: Floating Search Methods in Feature Selection. Pattern Recognition Letters 15:1119-1125, 1994). During the feature selection process, the putative features were added and removed in a floating sequential manner to thoroughly explore feature space and to maximize the diagnostic utility of the model. Specifically, an optimization function for biomarker selection was used to drive model diagnostic utility as measured by area under the receiver-operating characteristic curves (AUC ROC).

Machine learning: A low-order KPLS (see Rosipal R, Trejo LJ: Kernel Partial Least Squares Regression in Reproducing Kernel Hilbert Space. Journal of Machine Learning Research 2:97-123, 2002) machine learning methodology was used in all modeling rounds to provide a good compromise between rapid computation training time and a reasonably flexible space to be used for modeling, while significantly minimizing the number of parameters to be estimated compared to working directly with higher-order interactions.

For the KPLS learning method, two cost functions were considered for compatibility with the data and the machine learning methodology during an initialization phase. One was based on the AUC ROC. The other was based on the AUC ROC equally weighted with the sensitivity achieved at 80% specificity. The initialization phase was conducted using cross-validated training data from the first training fold to identify the cost function leading to the best learning rate and lowest false-positive rates. The false-positive rates were ascertained by appending false surrogate markers to the training set fold. The false surrogate markers were constructed using approximately half of the IHC marker data that was randomly reordered to be unrelated to the labeled outcome, while maintaining the same distributions. A false surrogate call rate was used to provide an indication of an inadequate cost function or insufficient training cross-validation during biomarker selection that was corrected and re-tested during the initialization phase.

The initialization phase indicated that, as the model was being grown, the AUC ROC cost function provided a lower false-positive rate than the sensitivity-weighted AUC ROC. Likewise, the AUC ROC appeared to be more suitable during the initial feature selection stages, leading to more rapid improvement in model performance. Therefore, the AUC ROC cost function was selected for feature selection.

Addition of Features to Base Model

Multiple rounds of modeling were performed. In the first round, the key composite components of Model 1A (e.g., see the expressions in the multivariate Cox model of the preliminary published model) were used as pseudovariables and combined with the new IHC marker data into a feature pool from which to build the model. Treatment types and subtypes were treated as coded variables to help produce a model that would be applicable to all patients.

Consistent with prior research and current prognostic models, such as the Nottingham Prognostic Index (NPI) and the Ninth (2005) St. Gallen Consensus (StG), the first round of modeling indicated that tumor diameter, tumor grade (grade), and continuous risk of age (age) were important features. They were consistently selected in high-scoring models throughout the modeling process. In addition, the previously identified IHC markers (ER, PGR, ERBB2, BCL2, and TP53) were chosen in both direct and pseudovariable forms during the five folds of feature selection. Tumor diameter, grade, and age were also selected.

In the second round of modeling, the individual features in the key composite components of Model 1A were treated as individual features rather than pseudovariables to ensure that the feature space was thoroughly explored and not biased by an early selection of a higher-order variable. The second round confirmed that tumor diameter, grade, and age were important features, as they were consistently incorporated into the model during the multiple rounds of naïve feature selection in addition to the core molecular features present in Model 1A.

In the third round of modeling, only HR-positive subjects with a poor prognosis based on Model1A were included to help identify the features most relevant to that group. In addition to the features described above, the IHC marker MTA1 was consistently incorporated into high-scoring models during feature selection. The absence of MTA1 in the previous two modeling rounds indicated that MTA1 interactions with the existing identified markers were likely complex, HR-dependent, and difficult to characterize in the mixed HR population, likely due to the size of the non-censored data used for the objective function.

In addition to the primary features described above, which consistently appeared in all of the modeling folds, the following features merited further investigation based on their frequent appearance in subsets of the modeling folds: IHC markers AURKA, AURKB, CKDN1B, high ER, GATA4, KRT5/6, KRT8/18, and TIMP1, and lymphovascular invasion (LVI). Several of these features fall under the invasion category of cancer markers, and other pathways, including cell cycle, differentiation, and transcription, are also represented. Such features will be critical to extend the current model, although they may only be indirect measures of risk and/or duplicative of informational content with respect to each other, the existing model, and/or the clinicopathologic factors.

Due to the fact that the new clinicopathologic and IHC markers were selected by machine learning, as described above, their potential interactions and specific contributions were unknown. We next sought to extend the model with the new features in a more transparent form while fully utilizing the dataset. Addition of the new features was done on Model 1B, the executable program form. Cox proportional hazards analysis was used to assess the significance and utility of the features in the context of the existing Model 1B, beginning with the clinicopathologic factors and then considering the newly identified IHC markers. The TriStar dataset was also included as a separate component of these analyses when appropriate for validation.

### Example IV

### Addition of New Clinicopathologic Features to Extend Model

Due to the very different baseline survival of HR-positive and HR-negative patients, the multivariate significance of each of the clinicopathologic features was first assessed with Model 1B using stratification by HR status. Cox proportional hazards analysis indicated that all of the newly identified features (tumor diameter, grade, age, and LVI) were highly statistically significant with p(Wald)<0.01. To further explore the interaction between these features and Model 1B, variations in the risks of the features were identified when factored by Model 1B prognosis, HR status, and each other. Risk due to grade, tumor diameter (pTd), age, and HR status were not statistically different (p[Wald] values of 0.63, 0.95, 0.28, and 0.95, respectively) in the Model 1B good prognosis (risk score ≦−0.3) vs. poor prognosis (risk score >−0.3) groups. Note: pTd represents pathologic tumor classes pT1-3, which are defined by tumor diameter, but not pT4, which is defined by tumor invasion. Thus, the relative risks of the clinicopathologic features are statistically independent. In combination, the above analyses indicated that all of the new clinicopathologic features were strong enough to be added to the model in a simple linear fashion.

Next, the new clinicopathologic features were assessed separately in the HR-positive and HR-negative subsets to determine whether interactions between clinicopathologic features and HR status needed to be addressed. This analysis indicated that there was an interaction between HR status and the clinicopathologic features. In the HR-positive IPC subpopulation, grade, pTd, age, and Model 1B were significant in a multivariate Cox proportional hazards analysis with p(Wald) values of 4.1E-4, 0.006, 7.1E-5, and 0.01, respectively. The overall p(logrank) value was 8.0E-10. However, in the HR-negative IPC subpopulation, only pTd (p=0.03) and age (p=0.05) were significant. The fact that grade (p=0.46) and Model 1B (p=0.31) were not significant in the HR-negative population is possibly due to the small sample size, but also indicates that there is likely a different interaction form within these factors than in the HR-positive population. Further, the general prognosis of the HR-negative patients was much worse than the HR-positive patients. Therefore, the HR-positive patients were analyzed separately from the HR-negative patients in subsequent analyses.

In the HR-positive patients, a similar Cox proportional hazards analysis, factoring grade, indicated that pTd risk is moderated by grade. When grade was 2 or 3, pTd factored by grade was significant with p=0.01 and p=7.3E-6, respectively. The weights in this Cox proportional hazards analysis indicated that pTd scaled nearly linearly with increasing grade in the IPC dataset. The square root of the tumor diameter (dsq) also has been reported as a risk factor in the literature. This was tested in the IPC dataset, and dsq was moderated by grade similar to pTd. Because dsq is a continuous variable, as opposed to the categorical nature of pTd, dsq is preferred. Because dsq scaled linearly with grade, it was added to the model as: dsq*grade.

We next determined whether the other newly identified clinicopathologic features were still statistically significant risk factors after dsq*grade had been added to the model. In the HR-positive subpopulation of the IPC dataset, LVI was a significant risk factor (p=0.04) in the context of age, HT status, Model 1B as a continuous variable in the good prognosis category (risk score≧−0.3), and dsq*grade when grade>1 (p values of 9E-6, 8E-3, 0.02, and 6E-6, respectively). Additional factor analysis on LVI in the IPC dataset indicated that invasion may pose sufficient risk to make HT a less relevant factor in predicting patient outcome (p=0.2 for HT in LVI-positive patients, and p=0.02 for HT in LVI-negative patients). Based on factor analysis, LVI also multiplies the risks of tumor diameter and grade, increasing the baseline risks by about 30% when present. Accordingly, the model form for the HR-positive subpopulation was chosen to be dsq*grade with LVI as an exacerbating factor and age as an additional factor to address death not due to disease within the overall survival data. As the risks of these clinicopathologic factors did not differ materially when assessed in the Model 1B good and poor prognosis groups, they were assumed to be additive to the molecular marker risks of Model 1B. Hence, the form of the extended model (Model 1C) using the clinicopathologic factors and Model 1B in HR-positive patients is a weighting of the clinicopathologic factors (here termed Clin_Base_HR-positive) in conjunction with Model 1B and a scaling factor:

Clin_Base_HR-positive=dsq*grade*(grade>1)*(0.065+0.03*LVI status)+age*.045) LVI status: 0, LVI-negative; 1, LVI-positive
Model 1C(HR-positive patient component)=f(Clin_Base_HR-positive, (Model 1B), A1)
A1 is a scaling factor that allows normalization and accounts for the relative contributions of the Clin_Base and Model 1B components.

In the HR-negative patients, the form of the interactions and parameter weights was also determined using the IPC dataset. Secondly, the resulting extended model (Model 1C) for HR-negative patients could be applied naïvely to the TriStar dataset, as the HR-negative subjects were not included in the development of the preliminary published model. The interactions between the clinicopathologic features were investigated with factored analysis. Factor analysis of age, Model 1B, and pTd with grade, indicated that pT'd risk was moderated by grade, which was significant for grade>1: grade 1 (p=1.0), grade 2 (p=0.03), and grade 3 (p=0.005). This factored covariate analysis also indicated that a good form of incorporating pTd and grade was a linear weighting of pTd for grade>1 with a weighted Model 1B risk score for subjects in the poor prognosis group. The impact of primary tumor invasion (pT4 in the TriStar patients) and LVI were assessed in conjunction with the other clinicopathologic factors in the HR-negative population, but were not found to be significant multivariate factors, or as moderating factors for pTd and grade. Likewise, age was not a significant factor in the HR-negative population.

Thus, in the HR-negative population, tumor diameter (in this case, pTd) and grade were significant factors in addition to Model 1B. This simple form was similar to that identified in the HR-positive population, except that the magnitude of grade did not appear to further increase the risk of death as it increased from 2 to 3. This may indicate that when a patient is HR-negative, only low grade is relevant, although small dataset effects must be considered. Likewise, the fact that age and LVI were not significant may be due to the risks of tumor diameter and grade superseding them, although this also may be a small dataset phenomenon. Similar to the formula for HR-positive patients, the form of the extended model (Model 1C) using the clinicopathologic factors and Model 1B in HR-negative patients is a weighting of the relevant clinicopathologic factors (here termed Clin_Base_HR-negative) in conjunction with Model 1B and a scaling factor:

Clin_Base_HR-negative=pTd*(grade>1)

Model 1C (HR-negative patient component)=f(Clin_Base_HR-negative, Model 1B, A2)

A2 is a scaling factor that allows normalization and accounts for the relative contributions of the Clin_Base and Model 1B components.

Next, the IHC markers identified during feature selection were analyzed using Cox proportional hazards analysis in the HR-positive subpopulation. For this analysis, Model 1C was used as a covariate with each of the IHC markers individually and with stratification by HT and CT. The molecular markers were analyzed as a continuous outcome, column 1, p(all), or by cut-points defined by their 1^{st} and 3^{rd} quartiles. The analysis was completed in the IPC dataset for subjects receiving RT and false discovery rate analysis was applied to account for multiple hypothesis testing. Table 4 indicates that TIMP1, MTA1, CDKN1B, and high ER have significant p values (<0.05), and MTA1 and CDKN1B have false discovery rates of less than 10%, indicating that they will likely generalize.

As CDKN1B was also available in the TriStar dataset, it was assessed to verify its model utility. Using the same analysis and cut-point (composite score>120) identified in the IPC dataset, CDKN1B was assessed and found to have significant utility in both the good and poor prognosis groups: hazard ratio=0.37 (95% Cl, 0.22-0.61), p=1.5E-3. The benefit of high CDKN1B was not statistically different between the good and bad prognosis groups, so CDKN1B was included as an additive factor to Model 1C to create Model 1D:

Model 1D=f(Model 1C, CDKN1B>120, A3)

A3 is a scaling factor that allows normalization and accounts for the relative contributions of Model 1C and CDKN1B.

Application of Model 1D to HR-positive patients

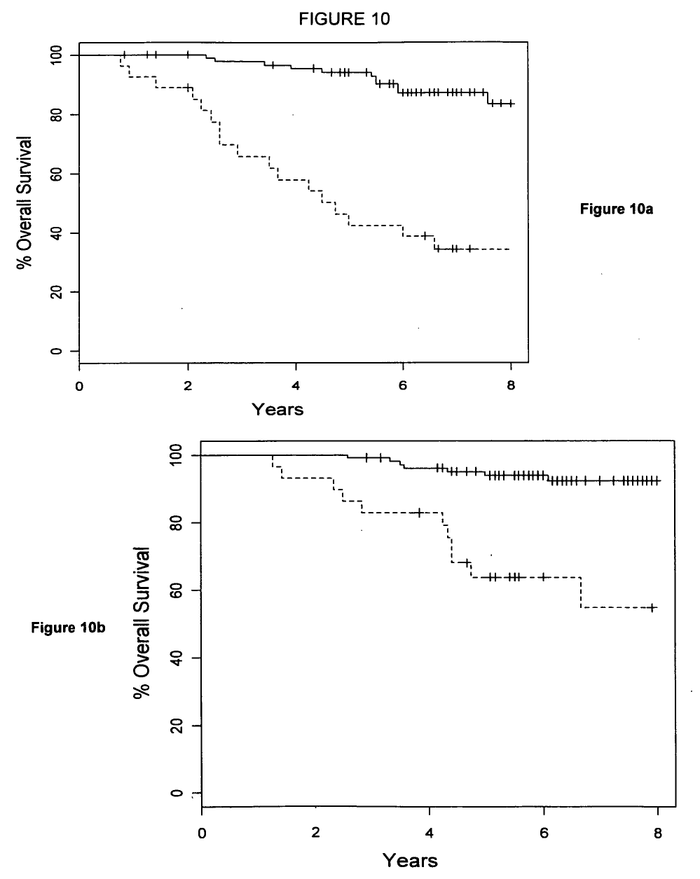

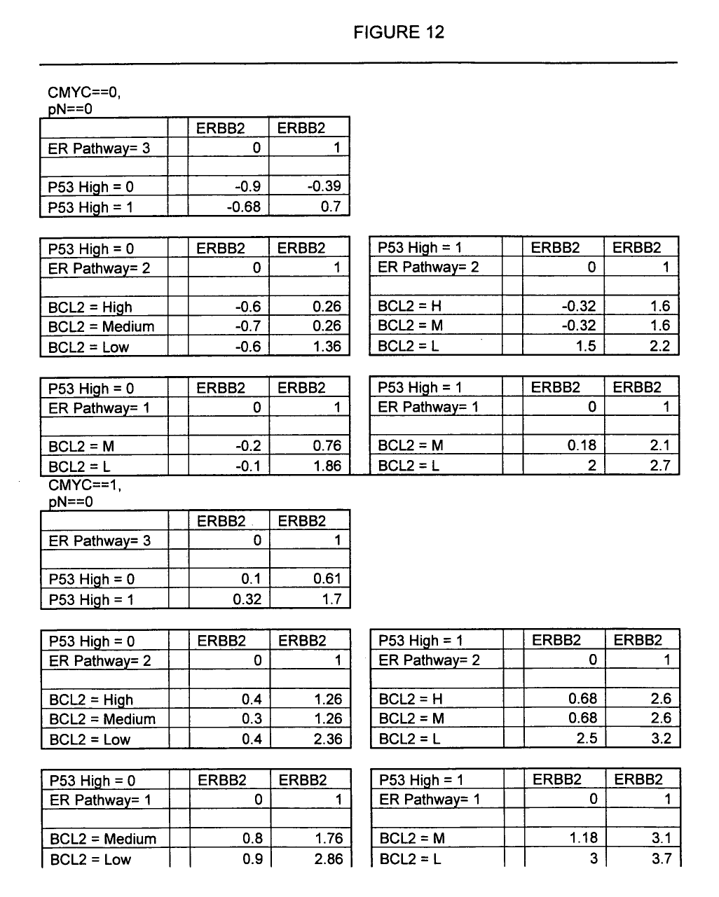

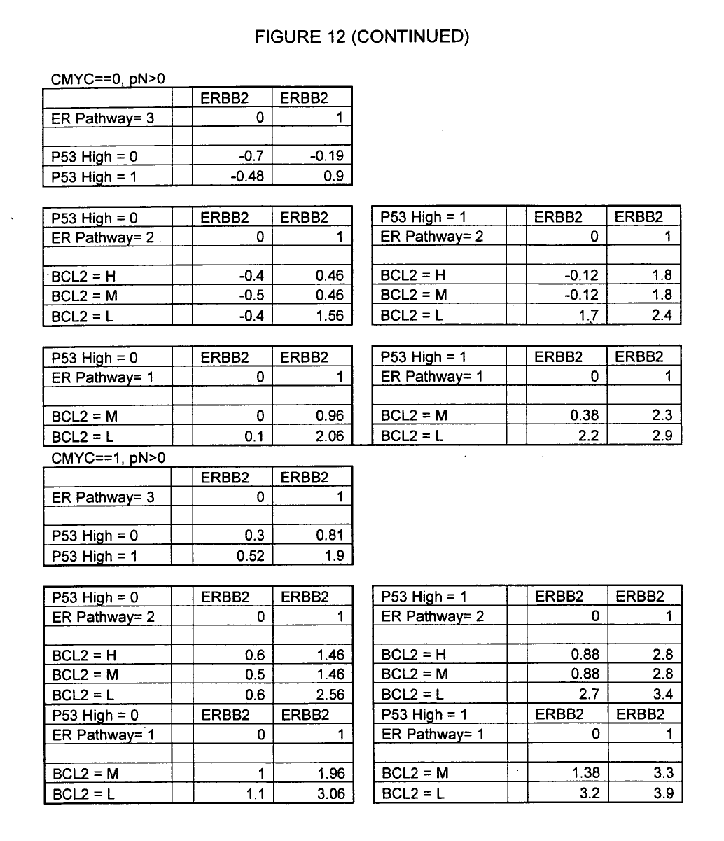

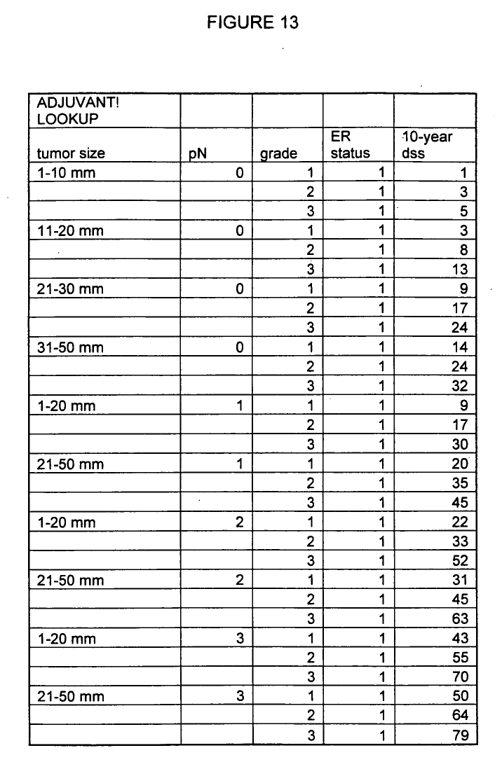

Model 1D, containing all of the new clinicopathologic (age, tumor diameter, grade, and LVI) and IHC marker (CDKN1B) features, was applied to HR-positive patients in each of the different treatment groups in the IPC dataset to demonstrate model utility using a risk score cut-point of 1.0. HT/CT/RT-treated patients from the TriStar dataset were combined with those in the IPC dataset, as they had never been used for model training. FIGS. 10-13 demonstrate that the model was statistically significant in the untreated, HT-only, and CT-only treatment groups. Similar to our previous findings, the Model 1D poor prognosis group showed a benefit with CT treatment (FIG. 14), whereas the good prognosis group did not (compare to FIGS. 10-13).

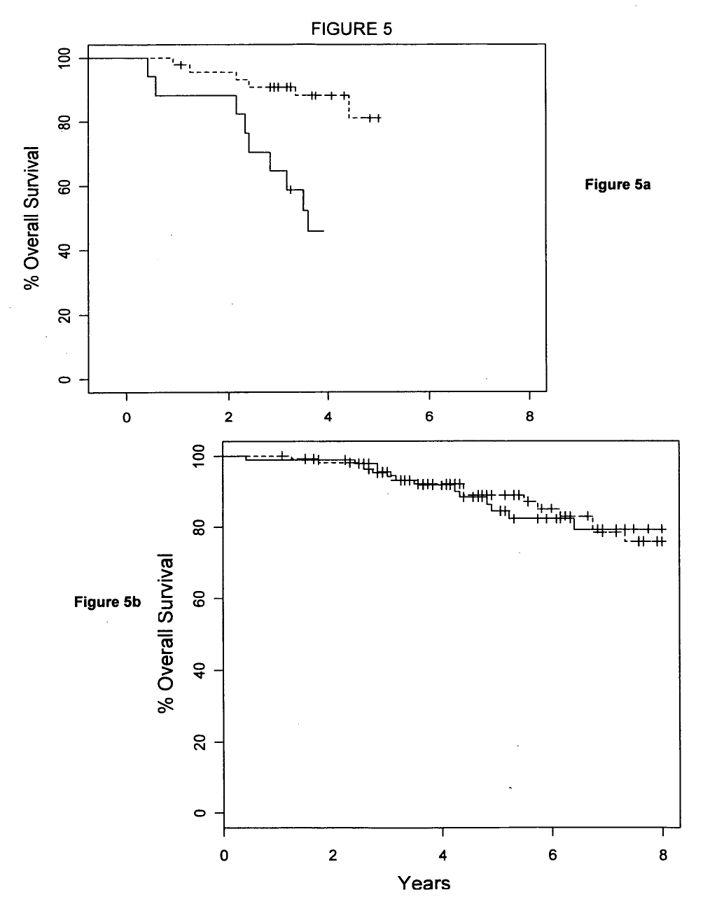

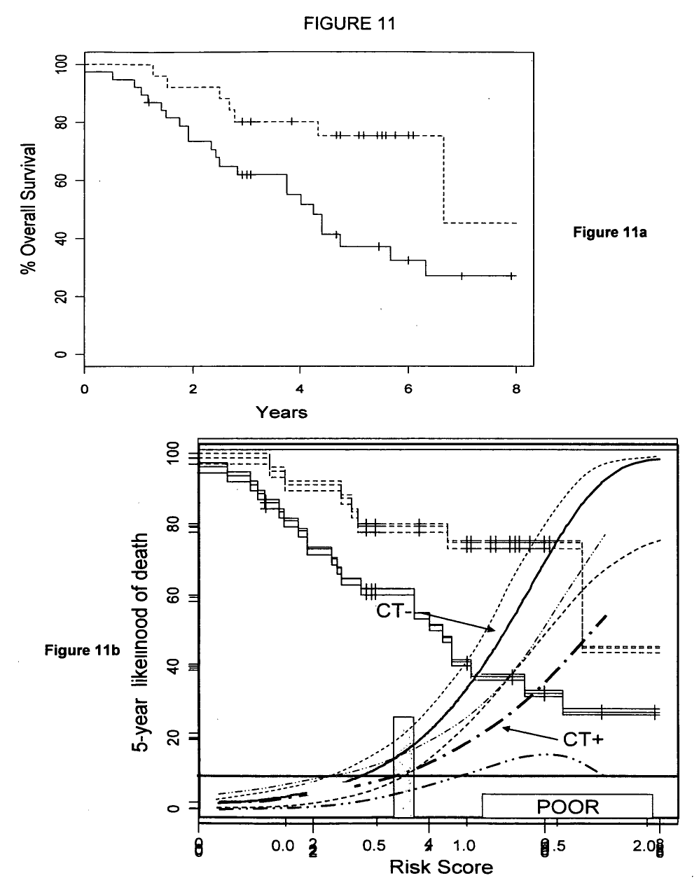

To demonstrate that the Model 1D has diagnostic utility beyond the standard models employed today, it was applied to the subset of the IPC population with an NPI score corresponding to an intermediate or high risk (>3.4). Model 1D was able to separate NPI>3.4 patients into good and poor prognosis groups in the subsets who did not receive HT (FIG. 15) or who received HT (FIG. 11), whether they received CT or not, shown in Table 5 (FIG. 5). Similar to the results shown in FIG. 14, the Model 1D poor prognosis group exhibited significant CT benefit within the NPI>3.4 group (FIG. 17). Likewise, Model 1D demonstrated significant diagnostic utility within the St. Gallen intermediate and high risk groups (FIGS. 18-20; Table 5 (FIG. 5)).

Model 1D was then assessed in the HR-negative patients by univariate Cox proportional hazards analysis as a continuous factor, as well as using a cut-point of the quartiles. As a continuous factor, Model 1D was significant (p=4E-5, n=86) in the IPC dataset with a multiplicative weight of 6.4. It was also significant in the combined HR-negative dataset from IPC and Tristar (p=3.7E-10, n=143) with a multiplicative weight of 6.6. The corresponding quartile analysis was done with a first-quartile cut-point of 0.08 and a third-quartile cut-point of 0.16. Analysis using the first quartile cut-point resulted in a hazard ratio of approximately 3 in the IPC dataset (p=0.01, n=86), and a hazard ratio of 3 in the combined IPC and Tristar datasets (p=7E-4, n=143).

Next, the utility of Model 1D was assessed in HR-negative patients within the elevated clinical risk (NPI>3.4 or St. Gallen>1) groups. For the NPI>3.4 analysis, Model 1D produced a hazard ratio of 2.3 (p=0.015, n=126). For the St. Gallen>1 analysis, Model 1D produced a hazard ratio of 2.6 (p=0.0024, n=133).

Finally, the effect of CT on HR-negative patients was assessed. In the total HR-negative subpopulation (regardless of risk score), there was an approximately 10% separation between the survival curves. However, CT benefit did not reach statistical significance in this subpopulation. In the Model 1D good prognosis group, CT benefit did not reach statistical significance at 5 years. However, in the Model 1D poor prognosis group, CT had a beneficial effect with a hazard ratio of 2.2 (p=0.0045, n=93). The survival curves exhibit a separation of approximately 30% at 6 years between the CT-treated and untreated groups.

### Example V

### Comprehensive Post-Modeling Assessment of the New Multi-Marker Model

Validation of IHC Markers in Preliminary Published Model

As described in Example II, all of the IHC markers in our previously published model developed in the TriStar dataset (ER, PGR, ERBB2, BCL2, and TP53) were also strong and highly statistically significant prognostic factors in the IPC dataset, providing independent validation of the preliminary published model.

Addition of Clinicopathologic Features

In addition, a series of standard statistical analyses conducted to determine the relevant model form for the clinicopathologic features showed that grade, tumor diameter, invasion (pT4 and LVI), and age (as a continuous risk factor) were all relevant in the HR-positive population, on top of pN status which was in the preliminary published model. In HR-negative patients, only grade and tumor diameter appeared to be relevant new clinicopathologic features.

While the general form of the interaction was similar between grade and tumor diameter in the HR-positive and HR-negative populations, the relative risks associated with diameter and grade were different. Thus, the weighting of these features in the model is different. Further, independent of the interactions between the clinicopathologic features, the risk associated with them was not moderated by the Model 1B risk score. Therefore, they were “additive” risks to the model. In a sense, HR status was the primary feature, and the clinicopathologic factors and remaining molecular markers were contextually dependent on the HR status. All of these features are well-established prognostic factors that appear in current clinical guidelines.

In order to minimize the number of parameters needed to be estimated to include the clinicopathological factors, and to preserve the IPC dataset as an independent dataset, Adjuvant! Online predictions of clinical outcome based on the SEER data base for Breast Cancer were used. These predictions indicate a risk of disease specific death in 10 years. To incorporate this factor into the model, the model risk and (adjuvant score)/20 were used as covariates in a cox proportional hazards analysis. The composite profile score was then model risk score plus a weighted function of the Adjuvant! Online Score.

Addition of IHC Markers

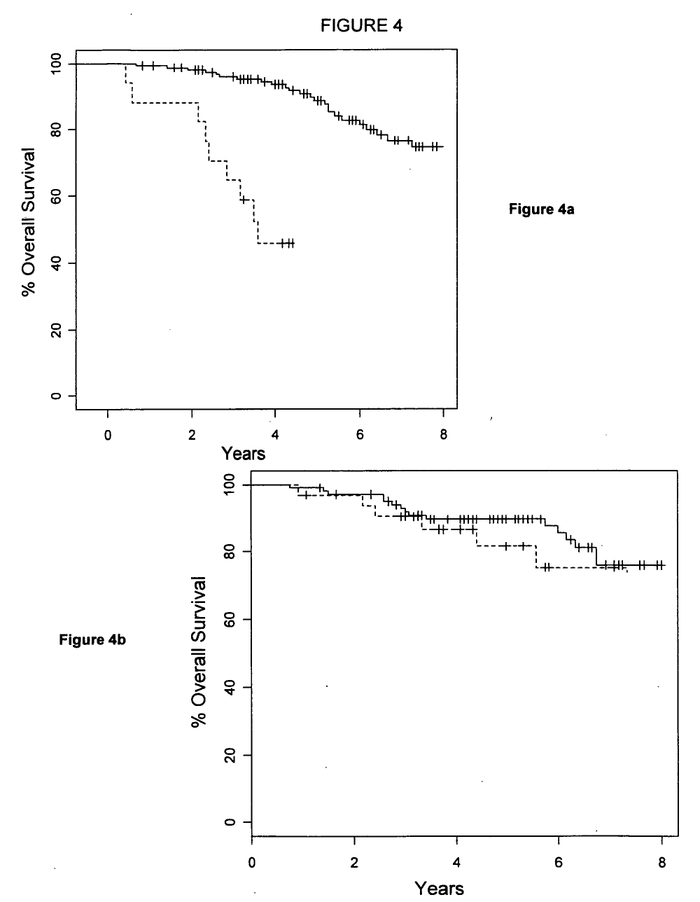

Univariate Cox proportional hazards and chi-squared analyses stratified by treatment types conducted on the new IHC markers indicated that CDKN1B and MTA1 were high-quality candidates for inclusion in the model, as high levels of these proteins were associated with better outcome. Multiple comparisons were accounted for by controlling false discovery rate with q-value analysis, limiting the number of false-positive identifications to ≦10% of the total number of factors deemed significant, as shown in FIG. 4 (Table 4). Because CDKN1B was available in the TriStar dataset, it was independently validated in the context of Model 1C and found to be an additive risk factor in the HR-positive population.

CDKN1B, also known as p27^{KiP1}, plays an anti-proliferative role carried out through inhibition of G1 cell cycle phase cyclin-dependent kinases. Although not all published reports in breast cancer show a statistically significant role, a preponderance of studies indicate that high levels of nuclear CDKN1B are associated with better prognosis and/or response to therapy (extensively reviewed in Alkarain A, Jordan R, Slingerland J: p27 deregulation in breast cancer: prognostic significance and implications for therapy. J Mammary Gland Biol Neoplasia 9:67-80, 2004 and Colozza M, et al: Proliferative markers as prognostic and predictive tools in early breast cancer: where are we now? Ann Oncol 16:1723-39, 2005). Similar to the new clinicopathologic features, CDKN1B was an independent linear factor (i.e., it did not interact to any large extent with the other features, indicating that it is in a distinct pathway, perhaps related to cell cycle).

MTA1, or “metastasis-associated 1” was originally identified as being over-expressed in metastatic rat mammary carcinoma cell lines in a differential screen with non-metastatic cells (Toh Y, Pencil S D, Nicolson G L: A novel candidate metastasis-associated gene, mtal, differentially expressed in highly metastatic mammary adenocarcinoma cell lines. cDNA cloning, expression, and protein analyses. J Biol Chem 269:22958-63, 1994). Subsequently, it was identified as an ER co-repressor that inhibits estrogen-induced transcription of genes with ER element-responsive promoters, perhaps through acetylation-dependent modulation of chromatin structure (Mazumdar A, et al: Transcriptional repression of oestrogen receptor by metastasis-associated protein 1 corepressor. Nat Cell Biol 3:30-7, 2001). Thus, the interaction we observed in this study with the hormone receptor pathway is not surprising. Most preliminary reports of MTA1 suggest that high levels are logically associated with worse prognosis. However, MTA1 is complicated in that it can be found in different isoforms in different cellular compartments and in different cell types, including normal epithelium, tumor cells, and stromal cells (Acconcia F, Kumar R: Signaling regulation of genomic and nongenomic functions of estrogen receptors. Cancer Lett 238:1-14, 2006). More recent reports indicate that it may be indicative of local invasion and lymph node metastasis, but not necessarily distant metastasis, and that tumors with the highest levels of MTA1 rarely undergo distant metastasis (Hofer M D, et al: Comprehensive analysis of the expression of the metastasis-associated gene 1 in human neoplastic tissue. Arch Pathol Lab Med 130:989-96, 2006). In fact, evidence indicates that elevated MTA1 sensitizes breast tumors to systemic therapies, such as tamoxifen (Martin M D, et al: Breast tumors that overexpress nuclear metastasis-associated 1 (MTA1) protein have high recurrence risks but enhanced responses to systemic therapies. Breast Cancer Res Treat 95:7-12, 2006). Furthermore, in prostate cancer (also a hormone-regulated disease), elevated MTA1 is associated with less frequent prostate specific antigen (PSA) recurrence after radical prostatectomy (Hofer M D, et al: The role of metastasis-associated protein 1 in prostate cancer progression. Cancer Res 64:825-9, 2004). The complex nature of the staining and interactions exhibited by this marker make it an excellent candidate for the IHC- and machine learning-based methodologies used in this continuing study.

Although “high ER” and TIMP1 did not achieve adjusted significance in the false discovery rate analysis, they did achieve individual statistical significance, making them excellent candidates for to include in a diagnostic panel, shown in Table 4 (FIG. 4). “High ER” (composite staining score>70—tentative threshold in the current study) is distinct from ER as it was included in the preliminary published study (composite staining score>0). Theoretically, patients who are positive for ER to any degree (>0) will have a negative risk score offset, whereas patients who are highly positive for ER may have a larger offset and/or differential interaction with other features. When ER levels were routinely assessed with quantitative ligand binding assays (LBA), studies indicated that responsiveness to hormone therapies directly correlated with levels of ER. There is evidence that when IHC assays became standard, the overall prediction of responsiveness was superior to LBA, but that the quantitative predictiveness became more questionable due to variations in tissue preservation, preparation, and staining. The latter issue may explain why “high ER” was not prognostic in the TriStar dataset and indicates that very strict standards must be established. These issues and relevant literature citations are discussed in Schnitt S J: Estrogen receptor testing of breast cancer in current clinical practice: what's the question? J Clin Oncol 24:1797-9, 2006.

TIMP1 is a member of a family of tissue inhibitors of matrix metalloproteinases (MMPs). The MMPs are a group of peptidases that degrade extracellular matrix (ECM), so elevated expression of their inhibitors, such as TIMP1, is expected to reduce invasiveness. Consistent with this hypothesis and with the findings of our study, overexpression of TIMP1 in breast carcinoma cells in vitro decreases their invasiveness (see for instance Dien J, et al: Signal transducers and activators of transcription-3 up-regulates tissue inhibitor of metalloproteinase-1 expression and decreases invasiveness of breast cancer. Am J Pathol 169:633-42, 2006), and elevated levels of TIMP1 assessed by IHC in breast tumors is associated with better prognosis (see for instance Nakopoulou L, et al: The favorable prognostic impact of tissue inhibitor of matrix metalloproteinases-1 protein overexpression in breast cancer cells. Apmis 111:1027-36, 2003).

Given the consistent identification of multiple invasion-related features (pT4, LVI, MTA1, and TIMP1), a series of univariate analyses were conducted on the other invasion IHC markers in the IPC dataset in Table 3 (FIG. 3). In this analysis, PLAU was also identified as a candidate for inclusion in the model with a false discovery rate of <10%. PLAU, also known as uPA or urokinase plasminogen activator, is a serine protease that is involved in the degradation of ECM as a member of the plasminogen activation system (PAS) pathway. The proteolytic activity of proteins in the plasminogen activation system (PAS) can degrade ECM either directly or through activation of matrix metalloproteinases (MMPs). In fact, MMP function may require PAS factors to enable intravasation (see for instance Kim J, Yu W, Kovalski K, Ossowski L: Requirement for specific proteases in cancer cell intravasation as revealed by a novel semiquantitative PCR-based assay. Cell 94:353-62, 1998), so the use of PAS components as tumor markers may be superior to MMPs. Other PAS proteins include PLAU's cell surface-associated receptor PLAUR (uPAR) (see for instance Andreasen P A, Kjoller L, Christensen L, Duffy M J: The urokinase-type plasminogen activator system in cancer metastasis: a review. Int J Cancer 72:1-22, 1997 and Schmitt M et al: Clinical impact of the plasminogen activation system in tumor invasion and metastasis: prognostic relevance and target for therapy. Thromb Haemost 78:285-96, 1997), as well as at least two of its inhibitors, SERPINE1 (PAI-1) and SERPINB2 (PAI-2).

Inclusion of the invasion-based IHC markers moves subjects from the poor prognosis group to the good prognosis group based on Model 1C, but they have little impact on patients who are already in the good prognosis group. Given the critical role of invasion (e.g., ECM degradation) in both local progression and distant metastasis, it is logical that the above markers are included in a modified model for certain subsets of patients, hereby identified in the instant invention.

Given the subjective nature of tumor grading, another area of interest is a molecular substitute for grade. Additional statistical analysis revealed that the IHC markers KRT5/6, KRT8/18, and MKI67 partially substitute for grade, although grade replaces them in multivariate analysis. As described above, other markers identified for future study include the centrosomal protein AURKA and AURKB, as well as the transcription factor GATA4.

Model 1D results

Given the ability to independently validate the CDKN1B finding in the TriStar dataset, Model 1D was created by adding CDKN1B and the new clinicopathologic features to the preliminary published model, and it was the focus of the remaining analyses (FIGS. 10-11). Adjuvant! Online predictions of clinical outcome based on the SEER data base for Breast Cancer were used to weight the effect of clinicopathological parameters. Model 1D was assessed in the NPI intermediate- and high-risk subpopulations, as well as the 2005 St. Gallen intermediate- and high-risk subpopulations. It maintained significant diagnostic utility in these subpopulations, and it was even useful in the HR-positive subpopulation not receiving HT. Thus, the selected molecular markers appear to represent not only those pathways directly related to HT response, but also related to the general aggressiveness of the cancer. This is also supported by the inclusion of CDKN1B as an independent linear factor, and the identification of markers related to invasion.

More specifically, high CDKN1B was identified as a positive prognostic factor, while low CDKN1B was a negative prognostic factor. However, given the role of CDKN1B in the cell cycle, negligible CCND1 was identified as an additional putative positive prognostic factor. The roles of CCND1 and CDKN1B in the cell cycle are in mutual opposition, CCND1 is required to advance in the cell cycle, while CDKN1B inhibits the cell cycle. In order for the cell cycle to progress, CCND1 must reach a critical threshold and form a complex necessary for advancement in the cell cycle. However, CKDN1B binds to CCND1 and in sufficient concentration inhibits the formation of this complex, halting cell cycle progression. Therefore either negligible CCND1 or high CDKN1B have a similar positive prognostic effect.

Recapitulation of the Invention

Breast cancer is the most common malignancy in Western women, and it is second only to lung cancer as the most common cause of cancer death. It affects millions of women worldwide. The current standard to decide on therapy is ER/PGR/ERBB2 status, but up to half of patients fail to respond. Accurate treatment outcome prediction arising from a test like this would guide patients to the most biologically and cost effective treatments in a timely fashion.

Historically patient data has been gathered in a series of immunohistochemical stains and/or fluorescent in situ hybridizations and/or other methods of molecular marker elucidation in a breast cancer patient's tumour and/or other tissue. In accordance with the present invention, the data gathered from these investigations was subjected to statistical analysis in combination with the patient's clinical and pathological data. The analysis is directed to revealing the patient's likelihood of suffering a recurrence of the cancer and/or other adverse events. Pathological data analysed included such features as the pathological status of the primary tumour and lymph nodes, the histological type and grade of the tumour cells, etc. Molecular markers analysed included BCL2, EGFR, ER, ERBB2, MYC, PGR, TP-53, CDKN1B, and over 50 others. The statistical analysis has also investigated assigning a patient to a sub-group(s) based on interdependencies of certain markers.

Thus the multivariate model of the present invention predicts outcomes based on statistically significant contributions of clinicopathological features and several molecular markers: ER, PGR, ERBB2, BCL2, TP-53, CDKN1B, MTA-1 and c-MYC gene amplification, among others. Analysis of additional molecular markers, such as ER coregulators such as AIB1, may further enhance this model.

The present invention will thus be realized to provide at least three separate and different insights, though not limited by such, as claimed below.

For example, the primary insight of the invention can be expressed by the statement:: “Ms. Patient, the aggressiveness of your breast cancer tumor is derived from considering a set of biomarkers in combination, and these biomarkers are the protein expression values of ER, PGR, BCL2, ERBB2, CDKN1B, CCND1, and TP-53, and c-MYC gene amplification, interpolated by an algorithm. Your personal probability of survival with adjuvant therapy only or without any therapy at all may be seen on this graph accompanying your test results.”

The secondary aspect of the invention can be expressed, by way of example, in the statement: “Ms. Patient, if you chose an adjuvant chemotherapy in addition to a treatment of endocrine therapy, your personal probability of survival may be seen on this graph accompanying your test results.”

The tertiary aspect of the invention can be expressed, by way of example, in the statement: “Ms. Patient, given that considering said biomarker panel has given you a low chance of long-term survival from your breast cancer using current treatment, you may want to consider a more aggressive course of treatment, including first-line use of an adjuvant targeted therapy in conjuction with or instead of said current treatment protocol, including investigational therapies.”

In accordance with these and still other insights obtained by the building, and the exercise, of the diagnostic test model in accordance with the present invention, the invention should be broadly defined by the following claims.

We claim:

1. A method of predicting response to adjuvant therapy or predicting disease progression in breast cancer, the method comprising:
obtaining a breast cancer test sample from a subject;
obtaining clinicopathological data from said breast cancer test sample;
analyzing the obtained breast cancer test sample for presence or amount of (1) one or more molecular markers of hormone receptor status, one or more growth factor receptor markers, and one or more tumor suppression/apoptosis molecular markers; (2) one or more additional molecular markers both proteomic and non-proteomic that are indicative of breast cancer disease processes consisting essentially of the group of angiogenesis, apoptosis, catenin/cadherin proliferation/differentiation, cell cycle processes, cell surface processes, cell-cell interaction, cell migration, centrosomal processes, cellular adhesion, cellular proliferation, cellular metastasis, invasion, cytoskeletal processes, ERBB2 interactions, estrogen co-receptors, growth factors and receptors, membrane/integrin/signal transduction, metastasis, oncogenes, proliferation, proliferation oncogenes, signal transduction, surface antigens and transcription factor molecular markers; and then
correlating (1) the presence or amount of said molecular markers and, with (2), clinicopathological data from said tissue sample other than the molecular markers of breast cancer disease processes, in order to deduce a probability of response to adjuvant therapy or future risk of disease progression in breast cancer for the subject.

2. The method according to claim 1 wherein the correlating in order to deduce a probability of response to a specific adjuvant therapy is of molecular markers drawn from the group consisting of
Chemotherapeutic Agents including 5-Fluorouracil, vinblastine, gemcitabine, methotrexate, goserelin, irinotecan, thiotepa, and topotecan;
Aromatase Inhibitors includng exomestane, anastrazole, and letrozole;
Ahti-estrogens including tamoxifen, fluvestrant, raloxifene, megestrol, or toremifene;
Taxanes including paclitaxol and docetaxel;
Antracyclines including doxurubicin and cyclophosphamide;
chemotherapy combinations including doxurubicin, cyclophosphamide, ocovorin, prednisone; and
targeted agents including. lapitinab, bevacizumab, trastuzumab, cetuximab, and panitumumab.

3. The method according to claim 1 wherein the correlating comprises:
determining the expression levels or mass spectrometry peak levels or mass-to-charge ratio(s) of one or more proteomic marker(s) and the numerical quantity of one or more clinicopathological marker(s) from breast cancer test sample excised from a patient population P1 before therapeutic treatment, clinical outcome C1 after a certain time period on said patient population P1 not being known in advance;
comparing said determined levels and numerical values to another set of expression levels or mass spectrometry peak levels or mass-to-charge ratio(s) of one or more proteomic marker(s) and the numerical quantity of one or more clinicopathological marker(s) from breast cancer test sample excised from a separate patient population P2 before therapeutic treatment, clinical outcome C2 after said certain time period on said patient population P2 being known in advance;
wherein the clinical outcome C1 and C2 is drawn from the group consisting essentially of: breast cancer disease diagnosis, disease prognosis, or treatment outcome or a combination of any two, three or four of these outcomes; and
training an algorithm to identify characteristic expression levels or mass spectrometry peak levels or mass-to-charge ratio(s) of one or more proteomic marker(s) and numerical quantity(ies) of one or more clinicopathological marker(s) between said patient population P1 and patient population P2 which correlate to clinical outcome C1 and clinical outcome C2, respectively.

4. The method according to claim 3 wherein the training of the algorithm on characteristic protein levels or patterns of differences includes the steps of
obtaining numerous examples of (i) said expression levels or mass spectrometry peak levels or mass-to-charge ratio(s) of one or more proteomic marker(s) and numerical quantity(ies) of one or more clinicopathological marker(s) data, and (ii) historical clinical results corresponding to this proteomic marker(s) and clinicopathological marker(s) data;
constructing an algorithm suitable to map (i) said characteristic proteomic and said clinicopathological marker(s) data values as inputs to the algorithm, to (ii) the historical clinical results as outputs of the algorithm;
exercising the constructed algorithm to so map (i) the said protein expression levels or mass spectrometry peak or mass-to-charge ratio(s) and clinicopathological marker(s) values as inputs to (ii) the historical clinical results as outputs; and
conducting an automated procedure to vary the mapping function inputs to outputs, of the constructed and exercised algorithm in order that, by minimizing an error measure of the mapping function, a more optimal algorithm mapping architecture is realized;
wherein realization of the more optimal algorithm mapping architecture, also known as feature selection, means that any irrelevant inputs are effectively excised, meaning that the more optimally mapping algorithm will substantially ignore specific proteomic marker(s) and specific clinicopathological marker(s) values that are irrelevant to output clinical results; and
wherein realization of the more optimal algorithm mapping architecture, also known as feature selection, also means that any relevant inputs are effectively identified, making that the more optimally mapping algorithm will serve to identify, and use, those input protein expression levels or mass spectrometry peak or mass-to-charge ratio(s) and said clinicopathological marker(s) values that are relevant, in combination, to output clinical results that would result in a clinical detection of disease, disease diagnosis, disease prognosis, or treatment outcome or a combination of any two, three or four of these actions.

5. The method according to claim 4 wherein the constructed algorithm is drawn from the group consisting essentially of:
linear or nonlinear regression algorithms;
linear or nonlinear classification algorithms;
ANOVA;
neural network algorithms;
genetic algorithms;
support vector machines algorithms;
hierarchical analysis or clustering algorithms;
hierarchical algorithms using decision trees;
kernel based machine algorithms such as kernel partial least squares algorithms,
kernel matching pursuit algorithms,
kernel fisher discriminate analysis algorithms, or kernel principal components analysis algorithms;
Bayesian probability function algorithms;
Markov Blanket algorithms;
a plurality of algorithms arranged in a committee network; and
forward floating search or backward floating search algorithms;
wherein the operation of each and of all algorithms can be shown in a look-up table.

6. The method according to claim 4 wherein the feature selection process employs an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms;
wherein operation of each and of all algorithms can be shown in a look-up table.

7. The method according to claim 4
wherein a tree algorithm is trained to reproduce the performance of another machine-learning classifier or regressor by enumerating the input space of said classifier or regressor to form a plurality of training examples sufficient (1) to span the input space of said classifier or regressor and (2) train the tree to emulate the performance of said classifier or regressor.

8. The method according to claim 2
wherein the correlating so as to predict the response to adjuvant therapy or disease progression is particularly so as to predict the response to chemotherapy or tumor aggressiveness respectively;
and wherein the method further comprises:
diagnosing breast cancer in a patient by taking a biopsy of breast cancer tissue and identifying that said biopsy is wholly or partially malignant;
identifying clinicopathological values associated with said malignant biopsy;
analyzing said malignant tissue for the proteomic markers ER, ERBB2, TP-53, BCL-2, CDKN1B, and c-MYC gene amplification, and one or more additional where proteomic markers;
evaluating the patient's prediction of response of said tumor to said therapy or evaluated risk of disease progression, respectively from said measured levels of proteomic markers and clinicopathological values; and
administering chemotherapy or other therapy as appropriate to the evaluated prediction of response of said tumor to said therapy or evaluated risk of disease progression, respectively.

9. The method according to claim 8 wherein the one or more additional proteomic markers includes, in addition to markers ER, ERBB2, TP-53, BCL-2, CDKN1B, and c-MYC gene amplification, one or more of the markers selected from the group that includes PGR, CCND1 and MTA1.

10. The method of claim 1 wherein the analyzing of one or more additional markers of breast cancer disease processes in addition to one or more molecular markers of hormone receptor status, one or more growth factor receptor markers, and one or more tumor suppression molecular markers is of one or more markers selected from the group consisting of two or more of the following: ESR1, PGR, ACTC, AIB1, ANGPT1, AURKA, AURKB, BCL-2, CAV1, CCND1, CCNE, CD44, CDH1, CDH3, COX2, CTNNA1, CTNNB1, CTSD, EGFR, ERBB2, ERBB2-ALT, ERBB3, ERBB4, EGFR, FGF2, FGFR1, FHIT, GATA3, GATA4, KRT14, KRT5/6, KRT8/18, KRT17, KRT19, MET, MKI67, MLLT4, MME, MMP9, MSN, MTA1, MUC1, MYC, NME1, NRG1, PARK2, PLAU, CDKN1B, S100, SCRIB, TACC1, TACC2, TACC3, THBS1, TIMP1, TP-53, VEGF, VIM or markers related thereto.

11. The method of claim 8
wherein the correlating is further so as to determine breast cancer treatment or prognostic outcome; and
wherein the correlating is performed in accordance with an algorithm drawn from the group consisting essentially of: linear or nonlinear regression algorithms;
linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.
wherein operation of each and of all algorithms can be shown in a look-up table.

12. The method of claim 11 wherein the correlating so as to further determine breast cancer treatment outcome is, in addition to prediction of response to chemotherapy, expanded to prediction of response to a targeted therapy.

13. The method of claim 1 wherein correlating is of clinicopathological data selected from a group consisting of Adjuvant! Online score, tumor nodal status, tumor grade, tumor size, tumor location, patient age, previous personal and/or familial history of breast cancer, previous personal and/or familial history of response to breast cancer therapy, and BRCA1&2 status.

14. The method of claim 1
wherein the analyzing is of both proteomic and clinicopathological markers; and
wherein the correlating is further so as to a clinical detection of disease, disease diagnosis, disease prognosis, or treatment outcome or a combination of any two, three or four of these actions.

15. The method of claim 1
wherein the obtaining of the test sample from the subject is of a test sample selected from the group consisting of fixed, paraffin-embedded tissue, breast cancer tissue biopsy, tissue microarray, fresh tumor tissue, fine needle aspirates, peritoneal fluid, ductal lavage and pleural fluid or a derivative thereof.

16. The method of claim 1
wherein the obtaining of the test sample from the subject before treatment of symptoms by a specific therapy; and
wherein the correlating is between (1) proteomic and clinicopathological marker values, and (2) the probability of present or future risk of a breast cancer progression for the subject or treatment outcome for said specific therapy, for a time period measured from the obtaining of said test sample chosen from the group consisting essentially of: 6, 12, 18, 24, 36, 60, 84, 120, or 180 months.

17. The method of claim 1
wherein the correlating is in accordance with an algorithm drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.
wherein operation of each and of all algorithms can be shown in a look-up table.

18. The method of claim 1
wherein the molecular markers of estrogen receptor status are ER and PGR, the molecular markers of growth factor receptors are ERBB2, the tumor suppression molecular markers are TP-53 and BCL-2; and the cell cycle molecular markers are CDKN1B and CCND1;
wherein the additional one or more molecular marker(s) is selected from the group consisting of essentially: c-MYC gene amplification, EGFR, AIB1, or KI-67;
wherein the correlating is by usage of a trained kernel partial least squares algorithm; and
the prediction is of time to recurrence when treated for breast cancer with a chemotherapeutic agent.

19. The method of claim 18
wherein the additional one or more molecular marker(s) is c-MYC gene amplification; and
wherein the chemotherapeutic agent is 5-Fluorouracil.

20. The method of claim 1
wherein the molecular markers of estrogen receptor status are ER and PGR, the molecular markers of growth factor receptors are ERBB2, the tumor suppression molecular markers are TP-53 and BCL-2; and the cell cycle molecular markers are CDKN1B and CCND1;
wherein and the additional one or more molecular marker(s) is selected from the group consisting of essentially: c-MYC gene amplification, EGFR, AlB1, pT4, LVI, PLAU, and TIMP1, or KI-67;
wherein the clinicopathological data is one or more datum values selected from the group consisting essentially of: Adjuvant! Online score, tumor size, nodal status, and grade; and
wherein the correlating is by usage of a trained kernel partial least squares algorithm; and the prediction is of risk of breast cancer progression.

21. The method of claim 20
wherein the additional one or more molecular marker(s) is c-MYC gene amplification; and
wherein the prediction is of risk of breast cancer progression as given by a likelyhood score derived from using Kaplan-Meier survival curves.

22. A kit comprising:
a panel of antibodies whose binding with breast cancer tumor samples has been correlated with breast cancer treatment outcome or patient prognosis;
one or more gene amplification assays corresponding to genes whose amplification has been correlated with breast cancer treatment outcome or patient prognosis;
reagents to assist said antibodies with binding to tumor samples; reagents to assist in determining gene amplification for genes whose amplification; and
a computer algorithm, residing on a computer, calculating in consideration of analyzed antibodies and amplified genes, interpolates from the aggregation of all binding values and level of gene amplifications upon the breast cancer tumor sample the prediction of treatment outcome for a specific treatment for breast cancer or future risk of breast cancer progression for the subject.

23. The kit according to claim 22 wherein the panel of antibodies comprises:
a poly- or monoclonal antibody specific for an individual protein or protein fragment and that binds one of said antibodies correlated with breast cancer treatment outcome or patient prognosis.

24. The kit according to claim 22 wherein the device comprises:
a number of immunohistochemistry assays equal to the number of antibodies and a number of gene amplication assays equal to the number of amplified genes.

25. The kit according to claim 22
wherein the antibodies are antibodies correlated with breast cancer treatment outcome, the gene amplification assays are for genes whose amplification is correlated with breast cancer treatment outcome, and the computer algorithm is an algorithm using kernel partial least squares or that is determined in accordance with a look-up table.

26. The kit according to claim 25
wherein the antibodies are antibodies specific to ER, PGR, ERBB2, TP-53, BCL-2, CDKN1B; and the gene amplification assay is for c-MYC.

27. The kit according to claim 25
wherein the treatment outcome is response to targeted therapy or chemotherapy.

28. The kit according to claim 22
wherein the antibodies are antibodies correlated with breast cancer progression, the gene amplification assays are for genes whose amplification is correlated with breast cancer treatment outcome, and the computer algorithm is an algorithm using kernel partial least squares or that is determined in accordance with a look-up table.

29. The kit according to claim 25
wherein the antibodies are antibodies specific to ER, PGR, ERBB2, TP-53, BCL-2, one or two of the markers selected from the group consisting essentially of CDKN1B and CCND1; and the gene amplification assay is for c-MYC.

30. The kit according to claim 28 wherein the antibodies are antibodies specific to ER, ERBB2, TP-53, BCL-2, CDKN1B, MTA-1, CCND1; and the gene amplification assay is for c-MYC.

31. The kit according to claim 28 wherein the antibodies are antibodies specific to ER, ERBB2, TP-53, BCL-2, CDKN1B, CCND1, one or more invasion markers selected from the group consisting essentially of pT4, LVI, PLAU, and TIMP1; and the gene amplification assay is for c-MYC.
